(19) United States
(12) Patent Application Publication
(10) Pub. No.: US 2026/0275279 A1
(43) Pub. Date: Sep. 17, 2026

(54) THYROCYTES AND USES THEREOF

(71) Applicant: LifeNet Health, Virginia Beach, VA (US)

(72) Inventors: Eda Rogers, Virginia Beach, VA (US); Kristina Wolf, Virginia Beach, VA (US); Jessica Weaver, Virginia Beach, VA (US); Erick Breathwaite, Virginia Beach, VA (US); Edward Lecluyse, Virginia Beach, VA (US); Sharon Presnell, Virginia Beach, VA (US); Jingsong Chen, Virginia Beach, VA (US); Justin Odanga, Virginia Beach, VA (US)

(73) Assignee: LifeNet Health, Virginia Beach, VA (US)

(21) Appl. No.: 19/164,020

(22) PCT Filed: Mar. 20, 2024
(86) PCT No.: PCT/US2024/020775

§ 371 Date: Sep. 10, 2025

Related U.S. Application Data
(60) Provisional application No. 63/453,356, filed on Mar. 20, 2023.
(60) Provisional application No. 63/560,476, filed on Mar. 1, 2024.
(60) Provisional application No. 63/562,777, filed on Mar. 8, 2024.

(51) Int. Cl. C12N 5/071; C12N 5/00

(52) U.S. Cl. CPC C12N 5/0617; C12N 5/562; C12N 2501/33; C12N 2501/36; C12N 2501/375; C12N 2501/39; C12N 2513/00; C12N 2533/90

(57) ABSTRACT

The present invention provides a thyrocyte product comprising viable thyrocytes isolated from one or more thyroid tissues, wherein the isolated thyrocytes have been cryopreserved. Also provided is a method for preparing a thyrocyte product. Further provided is a method for preparing a thyrocyte culture, comprising (a) seeding thyrocytes onto a surface in a culture vessel, wherein the thyrocytes are isolated from one or more thyroid tissues and have been cryopreserved; and (b) growing the thyrocytes in a growth medium, whereby a thyrocyte culture comprising cultured thyrocytes is prepared. The thyrocytes may form microtissues attached to the surface in a 3D culture. The cultured thyrocytes may form a monolayer in direct contact with the surface in a 2D culture. A method of testing drugs and other molecules for thyroid bioactivity is also provided.

### CROSS REFERENCES TO RELATED APPLICATIONS

This International PCT Application claims priority to U.S. Provisional Patent Application No. 63/562,777, filed Mar. 8, 2024, titled “THYROCYTES AND USES THEREOF,” the entire contents of which are incorporated herein by reference.

This International PCT Application further claims priority to U.S. Provisional Application No. 63/560,476, filed Mar. 1, 2024, titled “THYROCYTES AND USES THEREOF,” the entire contents of which are incorporated herein by reference.

This International PCT Application claims priority to U.S. Provisional Patent Application No. 63/453,356, filed Mar. 20, 2023, titled “THYROCYTES AND USES THEREOF,” the entire contents of which are incorporated herein by reference.

### FIELD OF THE INVENTION

The invention relates generally to isolated thyrocytes and thyrocyte cultures. The invention also relates to methods of producing and using the thyrocytes and thyrocyte cultures.

### BACKGROUND

Thyroid dysfunction is a leading endocrine disorder affecting approximately 200 million people worldwide. For example, in the US, 5% of the population has been diagnosed with an autoimmune thyroid disease (AITD). Examples of such diseases are Grave's disease and Hashimoto's thyroiditis. Graves' disease leads to hyperthyroidism, while Hashimoto's thyroiditis leads to hypothyroidism. Other thyroid diseases include thyroid tumors, thyroid cancer, postpartum thyroiditis for example. Besides iodine deficiency, which is also a cause of hypothyroidism, chemical exposure is another contributing factor of thyroid diseases. For example, a decrease in thyroid hormone synthesis could be caused by different chemicals like perchlorate, thiocyanate, methimazole, or 9-propyl-2-thiouracil. To address this problem, the U.S. Environmental Protection Agency (US-EPA) has developed a two-tiered Endocrine Disruptor Screening Program to screen for chemical molecules that could potentially lead to disruptions of endocrine system in human. The estimated cost to screen 1,200 pesticide, 2,500 pesticide inert ingredients, and 6,000 drinking water contaminants is around $1 million dollars, and the process could take up to 6 years. Outside of the U.S., the European Food Safety and Authority (EFSA) and the European Chemical Agency (ECHA) also have published a guidance on endocrine disruptor identification at the request of the European Commission. This on-going effort to screen potential thyroid disrupting compounds by different agencies has come with the need for a more high-throughput screening approach. Current test methods to identify new AITD, identify cures for thyroid diseases and also importantly, identify the effects of drugs and other compounds on the functioning of healthy and unhealthy thyroid are limited to the use of engineered cell lines and animal models; e.g. mouse, rat, vole, guinea pig, zebrafish, or sheep; reviewed in Patisaul et al, 2018 doi:10.1016/j.beem.2018.03.011.

Animal or cell line models are common test models for toxicological screening. However, these test models are far from being accurate representatives of human biological environment. In addition, animal-testing is costly and time consuming while cell lines often do not possess or rapidly lose their original characteristics after multiple passages. Therefore, there is a need for a system that is more physiologically relevant to human biological environment. There is a need for a physiologically relevant human thyroid model that mimics normal endocrine biology, as well as in some cases abnormal endocrine biology, which will allow for the reduction of animal testing in drug discovery and improve the translation of the in vitro test results to actual human response to chemicals, drugs, environmental stresses, etc.

### SUMMARY OF THE INVENTION

One of the problems to be solved by the present invention is to develop a test model using cryopreserved T4/T3/TG-competent primary human thyroid epithelial cells for compound screening. Further to this point, cryopreserved cells are needed in order to control experimental workflow and enable repeatable results across runs, and screening for perturbations in endocrine/thyroid function requires that the spectrum of thyroid hormones be measurable and responsive to stimuli and inhibitors (TG, T3, T4).

In one aspect, the present invention demonstrates that generation of hormone-competent thyrocytes—especially T4/T3-producing thyrocytes—includes the step of dissociation of thyroid tissue to generate an admixture of cell aggregates wherein the ratio of single cells:aggregates was at least about 29:1 and that the cellular composition was an admixture of both EPCAM+ thyrocytes (at least about 70%) and CD90+ fibrocytes (at least about 5% to about 30%). Regarding the CD90+ fibroblasts, it is important to understand that the culture of EpCAM+ epithelial cells in vitro may lead to the phenomonen of epithelial-mesenchymal transition, whereby epithelial cells will begin to concomitantly express markers associated with mesenchymal/stromal cells (such as CD90). Thus, it may be possible that the %-positive CD90 cells may exceed 30%; however, the % of CD90+ cells that do not concomitantly express EpCAM should remain 30% to 5%. In another aspect the present invention demonstrates that this thyroid cell population may be cryopreserved immediately after isolation (“p0” inventory) and subsequently used to initiate hormonogenic thyrocyte cultures for use in in vitro testing. In another aspect, the live dissociated thyrocytes (after enumeration using a fluorescent live/dead stain such as AO/PI and automated instrument such as a cellometer or flow cytometer) may be propagated in vitro while maintaining hormonogenic competency by following these steps: 1) seeding about 85,000/cm² live thyrocytes in at least a 29:1 single cell:aggregate ratio onto a tissue culture vessel surface; 2) maintaining the cells in culture for about 40 hours to about 6 days until they reach optimal confluence wherein optimal confluence is defined as exhibiting clear epithelial morphology with accumulation of cells in the z-plane of the culture without reaching full coverage of the tissue culture surface in the x-y plane, and 3) cryopreserving the propagated cells (“p1”) inventory. In step 2) the culture may be considered to be grown in a three dimensional space having mutually perpendicular x, y, and z directions, where the plane defined by x and y (x-y plane) is parallel to the culture vessel and the z direction is perpendicular to the x-y plane.

In another aspect, the invention demonstrates that the cryopreserved “p0” or “p1” thyrocytes may produce T4/T3/TG in a TSH-dependent manner (i.e., were competent for hormone production and suitable for use in detection of perturbations in hormone production) when the following conditions were met: 1) the thawed thyrocytes comprised an admixture of EPCAM+ epithelial cells (at least about 70%) and CD90+ fibrocytes (at least about 5% and up to about 30%); 2) the thyrocytes were seeded at a density of about 5000 to about 30000 cells/cm²; and 3) the culture environment supported the formation of multicellular tissues, or “microtissues” of at least about 30 microns (μm) in diameter up to about 200 microns (μm) in diameter through interaction of the cells with Matrigel or other suitable component that fosters cell aggregation and maintenance as a 3D structure with cell-cell contact.

As detailed in certain examples, the model was validated using native and antibody-based thyroid stimulating hormone receptor (TSHR) activators. In addition, the inhibitory effect of a TSHR inhibitor was also evaluated using two different approaches: therapeutic and preventive treatment. Results showed that both forms of the TSHR activators induced the production of thyroglobulin (TG) while TSHR inhibitor inhibited TG production in a dose-response manner. In one aspect, the disclosed system of preparing cryopreserved primary thyroid cells is a promising tool for compound screening because the cultured thyrocytes responded to various stimuli by exhibiting modified biological properties and/or function.

The present invention relates to thyrocytes isolated from a thyroid tissue, methods of making, and cultures thereof useful for screening an agent capable of modifying biological properties and/or functions of the cultured thyrocytes.

A thyrocyte product is provided. The thyrocyte product comprises viable thyrocytes isolated from one or more thyroid tissues. The isolated thyrocytes have been cryopreserved.

According to the thyrocyte product of the present invention, the isolated thyrocytes may be cultured for no more than 2 passages before being cryopreserved. The isolated thyrocytes may be primary cells. Primary cells refer to cells that are isolated or harvested directly from tissue or organs. The primary cells may be freshly isolated. The primary cells may be p0 cells. The primary cells may be freshly isolated cells and p0 cells. The isolated thyrocytes may have been cultured for 1 passage. The isolated thyrocytes may be positive for a biomarker selected from the group consisting of cytokeratin 7 (CK7), thyroglobulin (TG), epithelial cellular adhesion molecule (EpCAM), thyroid stimulating hormone (TSH) receptor and thyroperoxidase (TPO) receptor. At least 70% of the isolated thyrocytes may exhibit an epithelial morphology when cultured on a tissue culture surface in a monolayer. At least 70% of the isolated thyrocytes may exhibit an epithelial morphology when cultured on a tissue culture surface are positive for EpCAM by flow cytometry or immunochemistry

The thyrocyte product may further comprise additional cells in an amount of no more than 50% of the total number of the cells in the thyrocyte product. 1-90% of the isolated thyrocytes may be in aggregates.

According to the thyrocyte product of the present invention, the one or more thyroid tissues may be from one or more donors. Each of the one or more donors may be a human individual. The human individual may be healthy or may have one or more disease states.

The thyrocyte product may further comprise a cryopreservation medium. The cryopreservation medium may exclude an agent from an animal.

A first method for preparing a thyrocyte product is provided. The first thyrocyte product preparation method comprises (a) digesting one or more thyroid tissues with one or more enzymes, whereby one or more digested thyroid tissues are generated; (b) releasing thyrocytes from the one or more digested thyroid tissues, whereby released thyrocytes are generated; (c) seeding the released thyrocytes onto a surface in a culture vessel, whereby seeded thyrocytes are generated; (d) growing the seeded thyrocytes in a suitable medium, such as but not limited to an isolation or an expansion medium, whereby expanded thyrocytes are generated; and (e) harvesting the expanded thyrocytes before the expanded thyrocytes are cultured for more than 2 passages, whereby harvested thyrocytes are generated; (f) mixing the harvested thyrocytes with a cryopreservation medium to make a cryopreservation composition; and (g) freezing the cryopreservation composition. As a result, the thyrocyte product comprising viable thyrocytes is prepared.

According to the first method, the released thyrocytes may be an admixture of single cells and cell aggregates. A ratio of the number of single cells to the number of cell aggregates may be at least about 29:1. The released thyrocytes may comprise both EPCAM+ epithelial cells and CD90+ fibrocytes. In step (e), the thyrocytes may be harvested after about 40 hours to about 6 days of culture. In step (e), at the time of harvesting, the expanded thyrocytes may have an optimal confluence. As defined herein, optimal confluence means that the cells in culture exhibit an accumulation of epithelial cells in the z-direction while still displaying gaps of empty space in the x-y planes. The z-direction is perpendicular to the x-y plane. Typically, the x-y plane is on the culture surface. According to an aspect, the culture is located within a three-dimensional space having x-, y-, and z-directions, wherein the x- and y-directions form an x-y plane on the surface of the culture vessel and the z-direction is perpendicular to the x-y plane and the thyrocytes accumulate in the z-direction of the culture without reaching full coverage of the surface of the culture vessel in the x-y plane, and wherein the thyrocytes are grown for about 40 hours to about 6 days or until the at least 70% of the cells exhibit epithelial morphology.

A second method for preparing a thyrocyte product is provided. The second thyrocyte product preparation method comprises (a) digesting one or more thyroid tissues with one or more enzymes, whereby one or more digested thyroid tissues are generated; (b) releasing thyrocytes from the one or more digested thyroid tissues, whereby released thyrocytes are generated; (c) mixing the released thyrocytes with a cryopreservation medium to make a cryopreservation composition; and (d) freezing the cryopreservation composition, whereby the thyrocyte product comprising viable thyrocytes is prepared. The second thyrocyte product preparation method may further comprise (e) thawing the thyrocytes in the frozen cryopreservation composition; (f) seeding the thawed thyrocytes onto a surface in a culture vessel, whereby seeded thyrocytes are generated; (g) growing the seeded thyrocytes in a suitable culture medium, whereby expanded thyrocytes are generated; and (h) harvesting the expanded thyrocytes before the expanded thyrocytes are cultured for more than 2 passages, whereby harvested thyrocytes are generated.

According to the second method, the released thyrocytes may be an admixture of single cells and cell aggregates. A ratio of the number of single cells to the number of cell aggregates may be at least about 29:1. The released thyrocytes may comprise both EPCAM+ epithelial cells and CD90+ fibrocytes. In step (h), the thyrocytes may be harvested after about 40 hours to about 6 days of culture. In step (h), at the time of harvesting, the expanded thyrocytes may have an optimal confluence. As defined herein, optimal confluence means that the cells in culture exhibit an accumulation of epithelial cells in the z-direction while still displaying gaps of empty space in the x-y planes. The z-direction is perpendicular to the x-y plane. Typically, the x-y plane is on the culture surface. According to an aspect, the culture is located within a three-dimensional space having x-, y-, and z-directions, wherein the x- and y-directions form an x-y plane on the surface of the culture vessel and the z-direction is perpendicular to the x-y plane and the thyrocytes accumulate in the z-direction of the culture without reaching full coverage of the surface of the culture vessel in the x-y plane, and wherein the thyrocytes are grown for about 40 hours to about 6 days or until the at least 70% of the cells exhibit epithelial morphology.

According to the first or second thyrocyte product preparation method, the suitable medium may comprise one or more supplements comprising at least one of thyroid stimulating hormone (TSH), fetal bovine serum (FBS), penicillin-streptomycin (Pen-Strep), somatostatin (SMSTN), hydrocortisone (HDCN), growth hormone (GH), apo-transferrin (APOT), sodium iodide (NaI), sodium selenite (NaSel), L-glutathione reduced (GLUT), α-tocopherol (TOCO), DL-α-tocopherol acetate (TOCAC), insulin (INS), bovine thyroid stimulating hormone (bTSH), sodium bicarbonate, and a combination thereof. The suitable medium may comprise thyroid stimulating hormone (TSH). The TSH may be of bovine (bTSH) or human origin (hTSH); may be recombinant or isolated from whole tissue such as bovine or human pituitary gland. The suitable medium may comprise bovine thyroid stimulating hormone.

A method for preparing a thyrocyte culture is provided. The thyrocyte culture preparation method comprises (a) seeding thyrocytes onto a surface in a culture vessel, wherein the thyrocytes have been isolated from one or more thyroid tissues and cryopreserved; and (b) growing the thyrocytes in a suitable medium, whereby a thyrocyte culture comprising cultured thyrocytes is prepared.

According to the thyrocyte culture preparation method, the growth medium may comprise one or more supplements comprising at least one of fetal bovine serum (FBS), penicillin-streptomycin (Pen-Strep), somatostatin (SMSTN), hydrocortisone (HDCN), growth hormone (GH), apo-Transferrin (APOT), sodium iodide (NaI), sodium selenite (NaSel), L-Glutathione Reduced (GLUT), α-Tocopherol (TOCO), DL-α-Tocopherol acetate (TOCAC), insulin (INS), sodium bicarbonate and a combination thereof. The fetal bovine serum may be charcoal stripped fetal bovine serum (csFBS). The supplement comprising three or more of those components may be cryopreserved as a single solution prior to use in the media.

The thyrocyte culture preparation method may further comprise adding to the growth medium a biological response modifier that inhibits and/or stimulates and/or blocks and/or agonizes and/or antagonizes the TSH receptor or impacts any pathway of TG, T3, or T4 synthesis and/or degradation. Non-limiting examples of such modifiers may be growth factors, proteins, small molecules, antibodies or fragments thereof, antibody-drug conjugates, chemicals, or other living cells. Non-limiting examples are thyrocyte stimulating hormone (TSH), a stimulatory thyrotropin receptor antibody, a thyroid stimulating hormone receptor agonist, T cells or modified T cells (CAR-T), macrophages or other immune cell types, or a combination thereof.

The thyrocyte culture preparation method may further comprise adding one or more supplements to the growth medium, wherein the one or more supplements are selected from the group consisting of an Epidermal Growth Factor (EGF), a TGFβ inhibitor, a fibroblast growth factor (FGF) inhibitor, a Glycogen synthase kinase 3 (GSK-3) inhibitor, thyroid stimulating hormone (TSH), Rho-associated, coiled-coil containing protein kinase (ROCK) inhibitor, and a combination thereof.

According to the thyrocyte culture preparation method, the cultured thyrocytes may comprise a protein selected from the group consisting of cytokeratin 7 (CK7), thyroglobulin (TG), thyroid transcription factor 1 (NKX2-1), epithelial cellular adhesion molecule (EpCAM), thyroid stimulating hormone (TSH) receptor, and thyroperoxidase (TPO) receptor. The cultured thyrocytes may express a gene selected from the group consisting of thyroid stimulating hormone receptor (TSHR), thyroglobulin (TG), thyroperoxidase (TPO), Sodium Iodide Symporter (SLC5A5), Pendrin (SLC26A4), Dual Oxidase 1 (DUOX1), Dual Oxidase 2 (DUOX2), Dual Oxidase Maturation Factor 1 (DUOXA1) and Dual Oxidase Maturation Factor 2 (DUOXA2) genes.

According to the thyrocyte culture preparation method, the culture vessel may be selected from the group consisting of a dish, a chip, a microfluidic chip, an MPS (micro physiological system) a flask, a bead, a bioreactor, and a multi-well plate.

According to the thyrocyte culture preparation method, the one or more thyroid tissues may be from one or more donors. Each of the one or more donors may be a human individual. The human individual may be healthy. The human individual may suffer from a thyroid disease.

According to the thyrocyte culture preparation method, the thyrocytes may form microtissues attached to the surface. The resulting thyrocyte culture is also referred to as a 3D thyrocyte microtissue culture. Each of the microtissues may have a hollow space. The microtissues may have a diameter of 10-400 μm, or 20-300 μm, or 30-200 μm, or 40-200 μm, or 50-150 μm. The surface may be coated with an extracellular matrix (ECM). The microtissues may be in contact with the ECM. The microtissues may be in the ECM. In one aspect, the ECM encourages the aggregation of the cells to form aggregates and/or to maintain the cell aggregates that were either initially present and/or formed during the culturing step. Non-limiting examples of suitable ECMs include hydrogels, Matrigel (Corning) or HuBiogel (LifeNet Health). The surface may be coated with synthetic or naturally-occurring hydrogels that facilitate cell-cell interaction, non-limiting examples include gelatin, agarose, PDMS, Pluronics™, Poly-HEMA, or similar.

The 3D thyrocyte culture preparation method may further comprise producing a hormone selected from the group consisting of thyroxine (T4), 3,5,30-triiodothyronine (T3), monoiodothyronine (MIT) and diiodothyronine (DIT).

The 3D thyrocyte culture preparation method may further comprise exposing the thyrocytes to an agent. The agent may be therapeutic. The agent may perturb thyroid function. The 3D thyrocyte culture preparation method may further comprise (a) quantifying T4, T3, and or TG production in the thyrocyte culture before the exposing; and (b) quantifying T4 production in the thyrocyte culture after the exposing, and a change in T4, T3, and or TG production after the exposing as compared with that before the exposing indicates that the agent is a modifier of T4, T3, and or TG production. The agent may be selected from the group consisting of a chemical compound (including but not limited to drugs, environmental chemicals, food additives, crop protectants, etc.), a biological molecule, other living cells, T cells or modified T cells (CAR-T), macrophages or other immune cell types, and combinations thereof. The agent may be a therapeutic agent for treating a thyroid disease or disorder. The agent may be a therapeutic agent for preventing a thyroid disease or disorder.

According to the thyrocyte culture preparation method, the cultured thyrocytes may form a monolayer in direct contact with the surface. The resulting thyrocyte culture is also referred to as a 2D thyrocyte culture or monolayer culture. The surface may be coated with fibronectin or poly-lysine, or collagen type 1. In another aspect, the surface may not be coated with an extracellular matrix (ECM). Other non-limiting examples of suitable surface treatments that facilitate cell attachment include ECMs, plasma treatment, hydrophilic coatings, peptides, proteins, or fragments thereof, tissue extracts, etc.

The 2D thyrocyte culture preparation method may further comprise exposing the thyrocytes to an agent. The 2D thyrocyte culture preparation method may further comprise (a) quantifying thyroglobulin (TG), T3, and or TG production in the thyrocyte culture before the exposing; and (b) quantifying TG production in the thyrocyte culture after the exposing, and a change in TG production after exposing as compared with that before the exposing indicates that the agent is a modifier of TG production. According to an embodiment the method may comprise running parallel cultures such that one set of cultures is exposed to the agent and the other culture is not exposed to the agent. The TG production may be quantified by calculating a change in TG production between the two parallel cultures. The agent may be selected from the group consisting of a chemical compound, a biological molecule, and a combination thereof. The agent may be a therapeutic agent for treating a thyroid disease or disorder. The agent may be a therapeutic agent for preventing a thyroid disease or disorder.

For each 3D thyrocyte culture preparation method, a 3D thyrocyte culture prepared according to the method is provided.

For each 2D thyrocyte culture preparation method, a 2D thyrocyte culture prepared according to the method is provided.

### BRIEF DESCRIPTION OF THE DRAWINGS

FIG. 1 shows a schematic of cell production without culturing the cells prior to cryopreservation.

FIGS. 2A and 2B show photographs of “po” thyrocytes.

FIG. 3 shows the workflow for 3D culture described in Example 10.

FIG. 4 shows the workflow for the Thyroid Disrupting Chemical TDC assessment described in Example 11.

FIG. 5 shows a schematic representation of PHT (primary human thyrocytes) isolation and quality control in Example 12.

FIGS. 6A and 6B show the timeline of preventative (A) and therapeutic (B) treatment of cell cultures in Example 13.

FIG. 7 shows the reporting genes identification in Example 13.

FIG. 8 shows TG protein production as validation of additional thyrocyte lots in Example 13.

FIGS. 9A and 9B show TG production in the presence of TSHR activators in Example 13.

FIGS. 10A and 10B show TG production in a dose-response manner in Example 13.

FIGS. 11A, 11B, 11C, and 11D show comparison of K1-70 treatment conditions in Example 13.

FIG. 12 shows T4 production as a function of microtissue size.

FIG. 13 shows representative images of cell confluency on day 1 from flasks using either Trypan Blue (TB) staining or AOPI staining to determine cell seeding density. Lots 2318362, 2320634, and 2319504 are shown. Magnification at 4× (top row) and 10× (bottom row).

FIG. 14 shows Representative images of cell confluency on the day of cryopreservation from flasks using either Trypan Blue (TB) staining or AOPI staining to determine cell seeding density. Lots 2318362 and 2320634 are shown. Lot 2319504 was not done (ND) due to contamination. Magnification at 4× (top row) and 10× (bottom row).

FIG. 15 shows representative images of cell confluency on day 1 or 2 and the day of cryopreservation from flasks using AOPI staining to determine cell seeding density. Lots 2312660 and 2321028 are shown. Magnification at 4× (top row) and 10× (bottom row).

FIGS. 16A-D show morphology of cells and response to TSH.

FIGS. 17A-D show cell types, morphology and T4 production of thyrocytes according to an embodiment.

FIGS. 18A-E show microtissue formation in 3D culture.

FIGS. 19A-D show microtissue size and response to TSH.

FIGS. 20A-D show effect of TSH levels.

FIGS. 21-A-C show evaluation of thyroid-disrupting reference compounds.

FIG. 22 shows lack of correlation between TG and T4.

### DETAILED DESCRIPTION OF THE INVENTION

The present invention relates to viable thyrocytes isolated from a thyroid tissue, from which thyrocytes may have been cryopreserved. The inventors have surprisingly discovered thyrocyte cultures, especially 3D cultures, prepared with these isolated thyrocytes are biologically active, and provide an effective platform for screening an agent capable of modifying the biological activity of thyrocytes or a therapeutic for treating or preventing a thyroid disease or disorder.

The term “T4/T3/TG-competent” means that the thyrocytes are capable of producing and/or secreting and/or releasing at least one of these hormones.

The terms “thyrocytes,” “thyroid follicular cells,” and “thyroid epithelial cells” are used herein interchangeably and refer to epithelial cells in a thyroid gland that are responsible for production and secretion of thyroid hormones such as thyroxine (T4) and triiodothyronine (T3). The thyrocytes are positive for an epithelial marker, for example, cytokeratin 7 (CK7), thyroglobulin (TG), and epithelial cellular adhesion molecule (EpCAM). TG is a specific marker for T3/T4 expressing thyroid epithelial cells and this marker separates them from the other thyroid cell types.

The terms “fibrocyte,” “fibroblast”, and “stromal cell” are used interchangeably herein.

Thyrocytes isolated from a thyroid tissue are also referred to as isolated thyrocytes. The isolated thyrocytes may remain positive for an epithelial marker, for example, cytokeratin 7 (CK7), thyroglobulin (TG), and epithelial cellular adhesion molecule (EpCAM). Isolated thyrocytes may not produce thyroid hormones (e.g., T4 or T3).

The term “aggregates” as used herein refers to a cluster of two or more cells, each of which is in direct contact with at least another cell in the cluster. The cells in an aggregate may be of one or multiples cell types.

The term “2D thyrocyte culture” as used herein refers to a culture in which thyrocytes isolated from a thyroid tissue are seeded, grown in a suitable culture medium, and form a monolayer on a culture surface.

The term “confluence” used herein refers to a monolayer culture having complete cell-cell contact with substantially no visible gap or essentially no visible gap visible gap in the monolayer. “Confluence” may be recited herein in terms of percent coverage, meaning a percentage of the available area that is covered with cells having confluence. For the purpose of assessing thyrocyte cultures, an ideal thyrocyte culture that is used to generate p1 inventory should exhibit a multi-layered growth habit whereby the epithelial cells accumulate in the z-height of the culture, while the x-y planes exhibit gaps in coverage of the culture vessel, such that there is not 100% coverage (confluence) of the vessel surface. The x-y plane is understood to mean the plane of the plate upon which the culture is grown. The z-height (z-direction) is perpendicular to the x-y plane.

The term “3D thyrocyte culture” as used herein refers to a culture in which thyrocytes isolated from a thyroid tissue are seeded, grown in a suitable culture medium, and form microtissues on a culture surface.

The term “microtissue” used herein refers to three-dimensional (3D) spherical structures formed by isolated thyrocytes in a 3D culture. The microtissue may be formed by cultured thyrocytes on or in or in contact with an ECM rich environment provided by a substrate such as Matrigel, or HuBiogel, for example. The microtissue may have a lumen or partial lumen or lumen-like structure with hollow pockets.

Microtissues as described herein are multicellular tissues that are formed on Matrigel or other types of ECM such as HuBiogel. Suitable 3D multicellular structures or microtissues may be generated by various means and take slightly different forms while achieving the same goal of creating a polarized epithelial structure with a lumen or lumen-like structure or partial lumen that may accumulate colloid-like material in which thyroid hormone synthesis may occur. Similar 3D structures may include spheroids, organoids, neotissues, aggregates, clusters. Key features are cell-to-cell contact, epithelial cell polarization, and the formation of lumens, partial lumens, or lumen-like structures. The microtissues formed in or on or in contact with an ECM may have one or more features distinct from self-assembled spheroids. There are numerous means of fabricating or generating such 3D thyrocyte structures. Some cell types may form hollow structures (whether actually hollow, or only including a potential space) when aggregates are generated in spheroid form.

Microtissues may maintain some functions of the organ. The cells in microtissues may spontaneously self-organize and resemble their in vivo counterparts (e.g., bird-nest structures mimics the thyroid follicles). The structure may have internal lumens that may be formed within partial or complete spheroidal structures. The microtissues may be more irregularly shaped (not perfectly round) and they may or may not recapitulate features of the native thyroid follicle architecture in which epithelial cells surround a lumen filled with colloidal material.

The present invention provides a thyrocyte product. The thyrocyte product comprises viable thyrocytes isolated from one or more thyroid tissues. The isolated thyrocytes may have been cryopreserved. The thyrocyte product may have been isolated from thyroid tissue with cold ischemic times of 26 hours or less. For example, the thyroid tissue may have been isolated from thyroid tissue with cold ischemic times of 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, or 13 hours or less.

There is no particular limit to the number of cells per cryovial. The thyrocyte product may comprise about 0.1-10×10⁶, 0.1-5×10⁶, 0.5-10×10⁶, 0.5-5×10⁶ or 0.5-1.2×10⁶ viable cells per vial. The thyrocyte product may comprise about 0.1-2×10⁶, 0.1-3×10⁶, 0.5-4×10⁶, 0.5-8×10⁶ or 0.5-9×10⁶ viable cells per vial. The thyrocyte product may comprise about 0.1-3×10⁶, 0.2-3×10⁶, 0.3-3×10⁶, 0.4-3×10⁶ or 01-3×10⁶ viable cells per vial. The thyrocyte product may comprise about 0.1-5×10⁶, 0.1-5×10⁶, 0.5-5×10⁶, 0.5-5×10⁶ or 0.5-5×10⁶ viable cells per vial. The isolated thyrocytes may have been cryopreserved immediately after being isolated from the thyroid tissues. The isolated thyrocytes may have been cryopreserved after the isolated thyrocytes are cultured for expansion.

The isolated thyrocytes may not have been subjected to a viability enrichment step prior to initiation of culture. The isolated thyrocytes may not have been subjected to a viability enrichment step prior to initiation of cryopreservation. The isolated thyrocytes may not have been subjected to a density gradient step prior to initiation of culture. The isolated thyrocytes may not have been subjected to a density gradient step prior to initiation of cryopreservation.

Immediately post-isolation and prior to plating from pre-cryopreservation, the thyrocytes may be counted using acridine orange (AO) and propidium iodide (PI) with an automated instrument to measure the cell viability. According to an embodiment, the seeding density of the thyrocytes (viable cells/cm²) for seeding of the cells in tissue culture vessels after isolation and for culture prior to cryopreservation may be about 85,000 viable cells/cm² as measured using AO/PI and an automated counted method. For example the seeding density of the thyrocytes (viable cells/cm²) for seeding of the cells in tissue culture vessels after isolation and for culture prior to cryopreservation may be from 4000-1,000,000, or from 4000-950,000, or from 4000-900,000, or from 4000-850,000, or from 4000-800,000, or from 4000-750,000, or from 4000-700,000, or from 4000-650,000, or from 4000-600,000, or from 4000-550,000, or from 4000-500,000, or from 4000-450,000, or from 4000-400,000, or from 4000-350,000, or from 4000-300,000, or from 4000-250,000, or from 4000-200,000, or from 4000-150,000, or from 5000-125,000, or from 10,000-125,000, or from 25,000-125,000, or from 50,000-125,000, or from 75,000-100,000, or from 10,000-100,000, or from 10,000-250,000, or from 50,000-250,000 or from 50,000-200,000, or from 50,000-150,000, or from 75,000-250,000, or from 75,000-200,000, or from 75,000-150,000, or from 50,000-100,000, or from 75,000-90,000 viable cells/cm², as measured by counting using acridine orange (AO) and propidium iodide (PI) with an automated instrument to measure the cell viability. Immediately post-isolation and prior to plating from pre-cryopreservation, the thyrocytes may be counted using Trypan blue with an automated instrument to measure the cell viability. For example the seeding density of the thyrocytes (viable cells/cm²) for seeding of the cells in tissue culture vessels after isolation and for culture prior to cryopreservation may be from 4000-1,000,000, or from 4000-950,000, or from 4000-900,000, or from 4000-850,000, or from 4000-800,000, or from 4000-750,000, or from 4000-700,000, or from 4000-650,000, or from 4000-600,000, or from 4000-550,000, or from 4000-500,000, or from 4000-450,000, or from 4000-400,000, or from 4000-350,000, or from 4000-300,000, or from 4000-250,000, or from 4000-200,000, or from 4000-150,000, or from 5000-125,000, or from 10,000-125,000, or from 25,000-125,000, or from 50,000-125,000, or from 75,000-100,000, or from 10,000-100,000, or from 10,000-250,000, or from 50,000-250,000 or from 50,000-200,000, or from 50,000-150,000, or from 75,000-250,000, or from 75,000-200,000, or from 75,000-150,000, or from 50,000-100,000, or from 75,000-90,000 viable cells/cm², as measured by counting using trypan blue with an automated instrument to measure the cell viability.

The isolated thyrocytes have been cultured for no more than 0, 1, 2, 3, 4, 5, 6, 7, 8, 9, 10, 12, 14, 16, 18, 20, 25, or 30 passages, or no more than 1, 2, 3, 4, 5, 6, 7, 8, 9, 10, 12, 14, 16, 18, 20, 25, 30, or 60 cell doublings, before being cryopreserved. The isolated thyrocytes may be primary cells, i.e., not have been cultured. The isolated thyrocytes may have been cultured for 1 passage. The culture period length post-isolation, including the pre-harvest time and the pre-cryopreservation time may be no longer than about 6 days For example the culture period length post-isolation, including the pre-harvest time and the pre-cryopreservation time may be no longer than about 144, 142, 140, 138, 136, 134, 132, 130, 128, 126, 124, 122, 120, 118, 116, 114, 112, 110, 108, 106, 104, 102, 100, 98, 96, 94, 92, 90, 88, 86, 84, 82, 80, 78, 76, 74, 72, 70, 68, 66, 64, 62, 60, 58, 56, 54, 52, 50, 48, 46, 44, 42, 40, 38, 36, 34, 32, 30, 28, 26, or no longer than 24 hours. The culture period length post-isolation, including the pre-harvest time and the pre-cryopreservation time may be from 24 to 144 hours, or from 40 to 96 hours, or from 40 to 88 hours, or from 40 to 60 hours.

A passage number of a cell culture is the number of times the cultured cells have been sub-cultured, i.e., harvested and reseeded into one or more culture vessels each containing a fresh suitable medium to enable proliferation of the reseeded cells. The suitable medium may be a growth medium.

The thyrocytes seeded post isolation include multiple adherent and non-adherent cell populations. The “selected” adherent population after an initial expansion process contains primarily epithelial cells of interest, isolated thyrocytes. The condition may be adherence on tissue culture treated surfaces, not on any ECM, otherwise attachment of non-parenchymal cells occurs at a high rate. These isolated thyrocytes undergo physiological and functional changes after multiple passages.

As the passage number increases, the isolated thyrocytes may show reduced proliferative ability in a 2D culture, reduced ability to form microtissues in a 3D culture, and reduced functionality T4 and thyroglobulin (TG) production in a 3D culture.

The isolated thyrocytes may be positive for a biomarker selected from the group consisting of cytokeratin 7 (CK7), thyroglobulin (TG), epithelial cellular adhesion molecule (EpCAM), thyroid stimulating hormone (TSH) receptor and thyroperoxidase (TPO) receptor.

In the thyrocyte product, at least about 30%, 40%, 50%, 60%, 70%, 80%, 90% or 95%, or about 30-100%, 40%-100%, 50-100%, 60-100%, 70-100%, 80-100%, 90-100%, 30-95%, 40%-95%, 50-95%, 60-95%, 70-95%, 80-95% or 90-95% of the isolated thyrocytes exhibit an epithelial morphology when cultured on a tissue culture surface in monolayer.

The thyrocyte product may further comprise additional cells, i.e., cells that are not thyrocytes isolated from a thyroid tissue. The additional cells may comprise fibroblasts, endothelial cells (e.g., CD144+ cells), parafollicular cells expressing calcitonin hormone, but not thyroglobulin (TG). The additional cells may be in an amount no more than about 1%, 5%, 10%, 20%, 30%, 40%, 50%, 60%, 70%, 80% or 90%, or about 1-90%, 5-90%, 10-90%, 20-90%, 30-90%, 40-90%, 50-90%, 60-90%, 70-90%, 80-90%, 1-80%, 5-80%, 10-80%, 20-80%, 30-80%, 40-80%, 50-80%, 60-80%, 70-80%, 1-60%, 5-60%, 10-60%, 20-60%, 30-60%, 40-60% or 50-60% of the total number of the cells in the thyrocyte product.

In the thyrocyte product, about 1-90%, 5-90%, 10-90%, 20-90%, 30-90%, 40-90%, 50-90%, 60-90%, 70-90%, 80-90%, 1-80%, 5-80%, 10-80%, 20-80%, 30-80%, 40-80%, 50-80%, 60-80%, 70-80%, 1-60%, 5-60%, 10-60%, 20-60%, 30-60%, 40-60% or 50-60% of the isolated thyrocytes may be in aggregates. Each aggregate may have about 2-5, 2-10, 2-20 or 2-30 cells. The aggregates may have a diameter less than about 10, 50, 100, 200, 300, 400, 500 or 1,000 μm, or about 0.1-1, 0.1-10, 0.1-50, 0.1-100, 0.1-200, 0.1-300, 0.1-400, 0.1-500 or 0.1-1,000 μm.

The aggregates may be formed in an enzymatic tissue digestion and/or dissociation process. The microtissues may be formed in a more organized space in an ECM rich environment and upon stimulation with TSH, and may be more homogenous in shape and structure, that is, mostly exhibiting round shape and smooth/defined boundaries. The microtissues may include pockets which mimic colloid in the follicular lumen. Thyroxine hormones may be synthesized in the colloid of a thyroid tissue.

Freshly isolated cells from a thyroid tissue may represent a different combination of cell types from the expanded isolated cells before being cryopreserved, and may have different functional properties, for example, proliferation upon stimulation by TSH. It may be more advantageous to expand the freshly isolated cells to generate a more homogenous single cell suspension for more accurate quantification and better harvest yield of cells that selectively bind a hydrogel.

The one or more thyroid tissues may be from one or more donors, for example, from a single donor. Each donor may be a human individual or a non-human vertebrate (e.g., dog, monkey, sheep, mouse, rat, pig, etc.). The human individual may be healthy or suffer from a disease or disorder. The individual may have been exposed to certain chemicals that have a biological effect on the thyrocytes.

The thyrocyte product may further comprise a cryopreservation medium. The cryopreservation medium may exclude an agent from an animal, for example, fetal bovine serum (FBS) or human serum, a protein, an antibiotic, a hormone, or a growth factor. The cryopreservation medium may include a container or freezer in which the products are stored, such as a MedCo controlled-rate freezer (CRF) with NG5A solution.

The present invention also provides a first method for preparing a thyrocyte product. The thyrocyte product may be capable of releasing T4. In a preferred aspect, the thyrocyte product releases T4. In a preferred aspect, the thyrocyte product releases T3 and/or T4 in a TSH-responsive manner. The first thyrocyte product preparation method comprises (a) digesting one or more thyroid tissues with one or more enzymes in an isolation medium, whereby one or more digested thyroid tissues are generated; (b) releasing thyrocytes from the one or more digested thyroid tissues, whereby released thyrocytes are generated; (c) seeding the released thyrocytes onto a surface in a culture vessel, whereby seeded thyrocytes are generated; (d) growing the seeded thyrocytes in the isolation medium, whereby expanded thyrocytes are generated; and (e) harvesting the expanded thyrocytes before the expanded thyrocytes are cultured for 0, 1, 2, 3, 4, or 5 passages, whereby harvested thyrocytes are generated; (f) mixing the harvested thyrocytes with a cryopreservation medium to make a cryopreservation composition; and (g) freezing the cryopreservation composition, whereby the thyrocyte product comprising viable thyrocytes is prepared.

The present invention provides a second method for preparing a thyrocyte product. The thyrocyte product may be capable of producing T4. In a preferred aspect, the thyrocyte product releases T4. In a preferred aspect, the thyrocyte product releases T3 and/or T4 in a TSH-responsive manner. The second thyrocyte product preparation method comprises (a) digesting one or more thyroid tissues with one or more enzymes in an isolation medium, whereby one or more digested thyroid tissues are generated; (b) releasing thyrocytes from the one or more digested thyroid tissues, whereby released thyrocytes are generated; (c) mixing the released thyrocytes with a cryopreservation medium to make a cryopreservation composition; and (d) freezing the cryopreservation composition, whereby the thyrocyte product comprising viable thyrocytes is prepared. The second thyrocyte product preparation method may further comprise (e) thawing the thyrocytes in the frozen cryopreservation composition; (f) seeding the thawed thyrocytes onto a surface in a culture vessel, whereby seeded thyrocytes are generated; (g) growing the seeded thyrocytes in the isolation medium, whereby expanded thyrocytes are generated; and (h) harvesting the expanded thyrocytes before the expanded thyrocytes are cultured for more than 0, 1, 2, 3, 4, or 5 passages, whereby harvested thyrocytes are generated. The thawing step may include the use of a thawing medium followed by resuspension in an appropriate seeding medium.

According to some embodiments, in either thyrocyte preparation method, the thyrocytes may be cryopreserved immediately after isolation, with no pre-cryopreservation culture step. These cells may be referred to herein as “p0” inventory; “primary” or “initial” are other common terms for this type of cells.

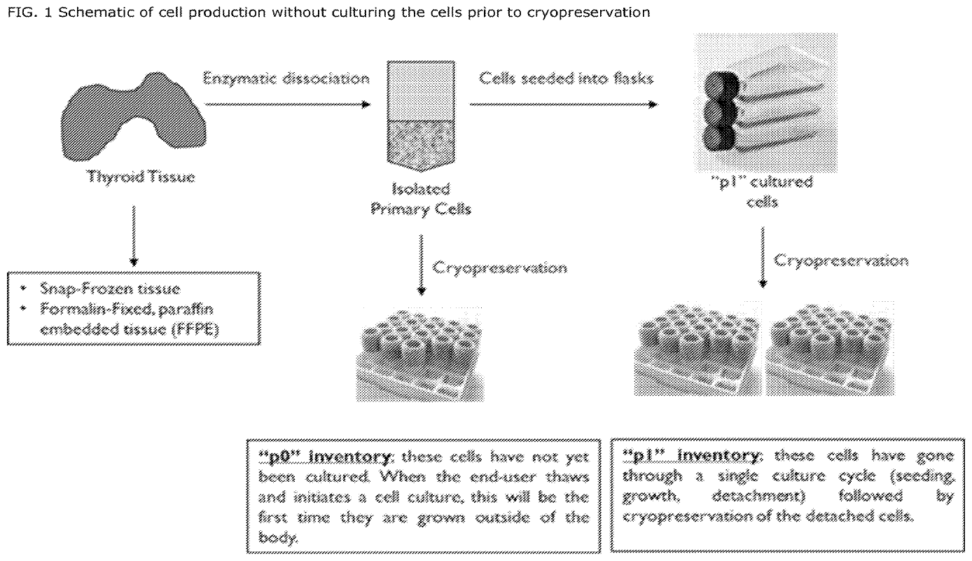

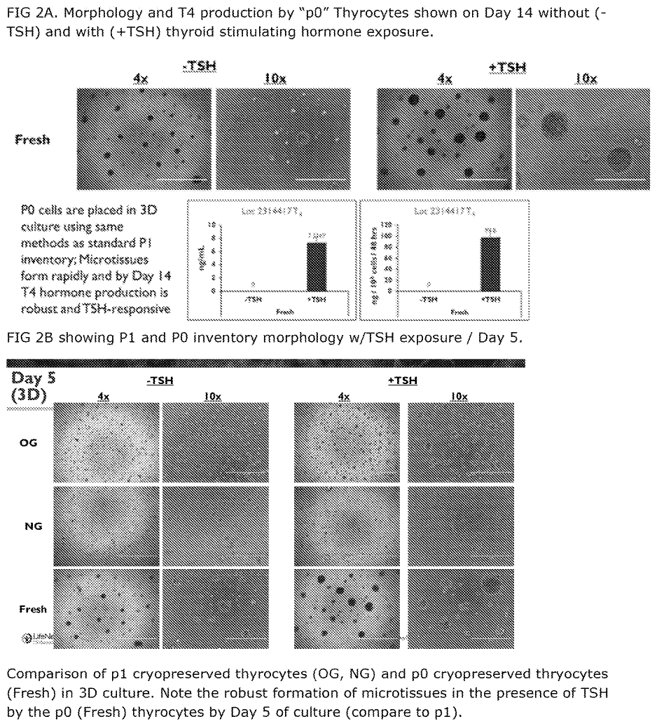

Conventional practice has involved isolating thyrocytes and placing them into culture prior to cryopreserving. However, according to one aspect of the present invention, it is feasible, and may sometimes be more desirable to directly cryopreserve the T4 thyrocyte population without a first culture step, which may better preserve cell phenotype and function as this enables direct-to-3D from cryopreserved state, having never been subjected to monolayer culture prior to initiation of the 3D culture. It should be appreciated that traditional monolayer culture on stiff surfaces such as polystyrene may cause gradual loss of function and in particular cause epithelial cells (like thyrocytes) to undergo epithelial-mesenchymal-transition (EMT) which may lead to the development of a mesenchymal fibroblast-like phenotype over time. This “p0” uncultured cryopreserved inventory may be advantageous in applications that require in vivo-like function and/or minimal manipulation of cellular components. This process is shown schematically in FIG. 1. Photographs of “p0” (non-cultured, cryopreserved) cells vs. “p1” cells which have been cultured through a single passage and then cryopreserved are shown in FIG. 2. FIG. 2 shows that p0 thyrocyte inventory forms 3D aggregates/microtissues when placed directly into 3D culture environment. T4 production is shown as robust, demonstrating that the culture step is not a requirement for generation of T4-competent cryopreserved inventory.

According to some embodiments, in either thyrocyte preparation method, in the digestion and/or the releasing step, the thyroid tissue may be dissociated to form an admixture of single cells and cell aggregates such that the single cell:aggregate ratio is greater than about 29:1. According to some embodiments, the single cell:aggregate ratio may be from about 29:1 to about 100:1 For example, the ratio may be from about 29:1 to about 90:1, or from about 29:1 to about 85:1, or from about 29:1 to about 80:1, or from about 29:1 to about 75:1, or from 29:1 to about 70:1, or from about 29:1 to about 65:1, or from about 29:1 to about 60:1, or from about 29:1 to about 55:1, or from about 29:1 to about 50:1, or from about 29:1 to about 45:1, or from about 29:1 to about 40:1, or from about 29:1 to about 35:1. According to some embodiments, the single cell:aggregate ratio may be from about 30:1 to about 100:1, or from about 35:1 to about 100:1, or from about 40:1 to about 100:1, or from about 45:1 to about 100:1, or from about 50:1 to about 100:1, or from about 55:1 to about 100:1, or from about 60:1 to about 75:1, or from about 65:1 to about 75:1, or from 70:1 to about 100:1, or from about 80:1 to about 100:, or from about 90:to about 100:1. In some embodiments, the single cell:aggregate ratio may be from about 30:1 to about 70:1, or from about 35:1 to about 65:1, or from about 40:1 to about 60:1, or from about 45:1 to about 55:1. According to another embodiment, the admixture of dissociated cells does not comprise solely single cells. Some embodiments demonstrate that exclusion of cell aggregates leads to loss or severe reduction in T4 production by the cells; retention of at least some portion of cell aggregates appears to facilitate retention of T4 production capacity in the cultured cells. Thus, when there are too many follicles vs. single cells, or there are too many single cells vs. follicles the resulting cells tend to lose or have reduced capacity for T4 production. Thus, ideally the dissociated admixture is not comprised solely of single cells or solely of aggregates, but has a single cell:aggregate ratio of about 29:1 or higher (up to about 75:1, or even up to about 100:1) as shown in the table below.

| Lot # | T4 Production | Single cell:follicle ratio |
|---|---|---|
| 2216507 | Yes | 69:1 |
| 2217737 | Yes | 52:1 |
| 2313606 | Yes | 38:1 |
| 2314417 | No | 34:1 and 9:1 |
| 2318942 | No | 28:1 |
| 2318957 | Yes | 36:1 |

According to another embodiment, if the thyrocytes are cultured prior to cryopreservation, the dissociated admixtures of single cell and aggregates may be placed into tissue culture vessels at a density of about 8.5×10⁴ cells/cm² with a supporting culture media.

According to the first or second thyrocyte product preparation method, the enzymes may be collagenase IA, collagenase IV, trypsin or a combination thereof. The digestion may be carried out in a buffer, for example, HBSS with calcium and magnesium. The thyroid tissues may have been treated with dispase in a transport solution to initiate dissociation.

According to the first or second thyrocyte product preparation method, the released thyrocytes may be in the form of single cells or aggregates. At least about 30%, 40%, 50%, 60%, 70%, 80%, 90% or 95%, or about 30-100%, 40%-100%, 50-100%, 60-100%, 70-100%, 80-100%, 90-100%, 30-95%, 40%-95%, 50-95%, 60-95%, 70-95%, 80-95% or 90-95% of the isolated thyrocytes exhibit an epithelial morphology when cultured on a tissue culture surface in monolayer. In the thyrocyte product, about 1-90%, 5-90%, 10-90%, 20-90%, 30-90%, 40-90%, 50-90%, 60-90%, 70-90%, 80-90%, 1-80%, 5-80%, 10-80%, 20-80%, 30-80%, 40-80%, 50-80%, 60-80%, 70-80%, 1-60%, 5-60%, 10-60%, 20-60%, 30-60%, 40-60% or 50-60% of the isolated thyrocytes may be in aggregates. Each aggregate may have about 2-5, 2-10, 2-20 or 2-30 cells. The aggregates may have a diameter less than about 10, 50, 100, 200, 300, 400, 500 or 1,000 μm, or about 0.1-1, 0.1-10, 0.1-50, 0.1-100, 0.1-200, 0.1-300, 0.1-400, 0.1-500 or 0.1-1,000 μm.

According to the first or second thyrocyte product preparation method, the seeded thyrocytes may be grown in the isolation medium until about 50-100%, 60-100%, 70-100%, 80-100% or 90-100% confluency.

According to the first or second thyrocyte product preparation method, the harvested cells may comprise at least about 30%, 40%, 50%, 60%, 70%, 80%, 90% or 95%, or about 30-100%, 40%-100%, 50-100%, 60-100%, 70-100%, 80-100%, 90-100%, 30-95%, 40%-95%, 50-95%, 60-95%, 70-95%, 80-95% or 90-95% epithelial cells. The harvested calls may include 60% or more of epithelial cells by CD326 and/or CK7/8 immunostaining as quantified by flow cytometry. The harvested cells may comprise additional cells, cells that are not thyrocytes isolated from a thyroid tissue. The additional cells may comprise fibroblasts, endothelial cells (e.g., CD144+ cells), parafollicular cells expressing calcitonin hormone, but not thyroglobulin (TG). The additional cells may be in an amount of no more than about 1%, 5%, 10%, 20%, 30%, 40%, 50%, 60%, 70%, 80% or 90%, or about 1-90%, 5-90%, 10-90%, 20-90%, 30-90%, 40-90%, 50-90%, 60-90%, 70-90%, 80-90%, 1-80%, 5-80%, 10-80%, 20-80%, 30-80%, 40-80%, 50-80%, 60-80%, 70-80%, 1-60%, 5-60%, 10-60%, 20-60%, 30-60%, 40-60% or 50-60% of the total number of the cells in the thyrocyte product. According to an embodiment, the cells may comprise less than about 5% endothelial cells. According to an embodiment, the cells may comprise from about 5% to about 30% fibroblasts as measured by CD90 or FSP1 by either flow cytometry or immunocytochemistry. According to an embodiment the harvested cells may be an admixture of thyrocytes, in which at least about 75% of the cells are EPCAM+ and the remaining population comprises non-epithelial cells or cells that simultaneously express both EPCAM (CD326) and one or more of CD90 and FSP1. According to an embodiment, the harvested cells may be used in vitro or in vivo for the TSH-regulated production of thyroid hormones including T3 and T4. According to an embodiment, the T4-competent human thyrocyte population may comprise at least about 70% EPCAM+ epithelial cells, and further may comprise at least 5% CD90 or FSP1+ thyroid fibroblasts. According to some embodiments the thyrocytes may include at least about 1 to 10% fibroblasts. For example, the thyrocyte population may include at about 1, 2, 3, 4, 5, 6, 7, 8, or 10% fibroblasts. According to some embodiments the thyrocytes may include less than about 50% of CD90+ cells. According to some embodiments the thyrocytes prepared according to the first or the second method may be used immediately for further experiments, including 3D cell culture, 2D cell culture, or other analyses; these thyrocytes may be cryopreserved for future use.

According to the first or second thyrocyte product preparation method, the T4-competent human thyrocytes may be capable of forming microtissues/3D cell aggregates/follicle-like structures under suitable culture conditions that support 3D cell growth. This may include culturing in or on hydrogels, 3D scaffolds, hanging drops or low-binding cultureware or other approaches that facilitate the formation of 3D cellular structures comprising polarized thyrocytes and the formation of lumen-like structures. According to some embodiments, the T4-competent thyrocytes configured as three-dimensional multicellular structures, each structure being at least about 30 microns in diameter up to about 200 microns in diameter. Each structure may comprise polarized human T4-competent thyrocytes. Each structure may comprise a lumen-like element surrounded or partially surrounded by polarized human thyrocytes. As used herein the term “T4 competent thyrocyte” means that the thyrocyte or aggregate thereof is capable of producing at least T4 among the thyroid hormones. It is not limited to the thyrocytes that are capable of only producing T4.

According to an embodiment, a 3D human thyrocyte culture system is provided. The T4-competent 3D human thyrocyte culture system may comprise the following elements. T4-competent human thyrocytes that are isolated by the first or the second method and cryopreserved after a single cycle of in vitro culture as described in above, stored for at least 3 days at temperatures at or below −20° C. and then thawed. Ideally, the cryopreserved thyrocytes should be stored at or below −150° C. According to another embodiment, the thyrocytes may be prepared according to the first or the second method and then cryopreserved immediately after isolation as described in the first or the second method, i.e., NOT having been placed in in vitro culture and then stored for at least 3 days at temperatures at or below −20° C. and then thawed. According to another method, the thyrocytes are freshly prepared (NOT having been cryopreserved at any point after isolation) according to the isolation process described in the first or the second method.

According to an embodiment of the first or second method, the thyrocyte product may comprise cell aggregates (a.k.a. “clusters”, “microtissues”, “spheroids”, “organoids”) that are at least about 30 microns in diameter to at most about 200 microns in diameter; the aggregates may comprise polarized thyroid epithelial cells. The thyrocytes may comprise a lumen-like structure that is surrounded or partially surrounded by polarized thyroid epithelial cells.

According to an embodiment, the thyrocyte product may comprise a hydrogel, scaffold, device, or biomaterial that supports cell aggregation and/or “microtissue” formation and/or spheroid formation (i.e., any conformation that enables thyrocytes to assemble into a structure comprising polarized thyrocytes and a lumen-like structure that is surrounded or partially surrounded by polarized thyrocytes). Non-limiting examples include: low-binding tissue culture vessels that repel cell-vessel interactions and support cell-cell interactions, devices that support hanging drop cell aggregate formation, hydrogels such as Matrigel, reduced growth factor Matrigel, Geltrex™, HuGentra™, HuBiogel™, Gelatin, extracellular matrix molecules whether extracted from native tissue or recombinant, collagen, alginate, hyaluronic acid; non-fouling coatings such as poly(2-hydroxymethyl methacrylate) (pHEMA), non-ionic triblock copolymers (poloxamers) such as Pluronic™, Kolliphor™ or Synperonic™, polydimethylsiloxane (PDMS), polyethylene glycol (PEG); porous biocompatible material scaffolds including natural and/or synthetic-origin materials; synthetic or naturally-occurring peptides, or combinations thereof. In some embodiments, multiple materials may be used concomitantly or serially to support thyrocyte cultures.

According to the first or second thyrocyte product preparation method, the thyrocyte product may comprise a suitable culture media in contact with the thyrocyte product. The suitable culture media may comprise thyroid stimulating hormone at concentrations from about 0.003 mIU/mL to about 3.0 mIU/mL. According to an embodiment, the culture media may be generated by adding a frozen multi-component supplement to a base media, wherein the supplement comprises 3-11 of the following elements: Thyroid Stimulating Hormone (TSH), Insulin, Somatostatin, Hydrocortisone, Growth Hormone, Apotransferrin, Sodium Iodide, Sodium Selenite, Glutathione, Vitamin E (alpha-Tocopherol), DL-alpha Tocopherol Acetate. According to an embodiment, the frozen supplement may comprise up to 10 elements from the list above but specifically does NOT include TSH.

According to the first or second thyrocyte product preparation method, the thyrocyte product may comprise about 0.1-10×10⁶, 0.1-5×10⁶, 0.5-10×10⁶, 0.5-5×10⁶ or 0.5-1.2×10⁶ viable cells per cryovial. The thyrocyte product may comprise about 0.1-2×10⁶, 0.1-2×10⁶, 0.5-2×10⁶, 0.5-2×10⁶ or 0.5-2×10⁶ viable cells. The thyrocyte product may comprise about 0.1-3×10⁶, 0.1-3×10⁶, 0.5-3×10⁶, 0.5-3×10⁶ or 0.5-3×10⁶ viable cells. The thyrocyte product may comprise about 0.1-5×10⁶, 0.1-5×10⁶, 0.5-5×10⁶, 0.5-5×10⁶ or 0.5-5×10⁶ viable cells.

According to the first or second thyrocyte product preparation method, the isolation medium may comprise one or more supplements selected from the group consisting of fetal bovine serum (FBS) or human serum, penicillin-streptomycin (Pen-Strep), somatostatin (SMSTN), hydrocortisone (HDCN), growth hormone (GH), apo-transferrin (APOT), sodium iodide (NaI), sodium selenite (NaSel), L-glutathione reduced (GLUT), α-tocopherol (TOCO), DL-α-tocopherol acetate (TOCAC), insulin (INS), bovine thyroid stimulating hormone (bTSH), sodium bicarbonate, and a combination thereof. For example, the isolation medium may comprise bovine thyroid stimulating hormone (bTSH), or TSH from other species, or recombinant. According to an embodiment, the isolation and/or culture medium may be prepared by adding to Coon's modified Ham's F-12 base medium a pre-frozen supplement including at least one of human recombinant insulin, somatostatin, hydrocortisone, growth hormone, apotransferrin, sodium iodide, sodium selenite, reduced L-glutathione, alpha-tocopherol, alpha-tocopherol acetate, sodium bicarbonate and optionally including one or more of: fetal bovine serum, charcoal-stripped fetal bovine serum, and/or Thyroid Stimulating Hormone (TSH). According to an embodiment, the isolation and/or culture medium may be prepared by thawing a pre-frozen mixture of three or more of the following components: Coon's modified Ham's F-12 base medium, human recombinant insulin, somatostatin, hydrocortisone, growth hormone, apotransferrin, sodium iodide, sodium selenite, reduced L-glutathione, alpha-tocopherol, alpha-tocopherol acetate, sodium bicarbonate and optionally comprising one or more of: fetal bovine serum, charcoal-stripped fetal bovine serum, and/or Thyroid Stimulating Hormone. According to another embodiment, TSH may not be one of the components that is added. Components not added to the pre-frozen mixture may be added separately at the time of thaw/preparation for use.

The ratio of single cells vs aggregates in the thyrocyte product may be affected by the initial digestion, filtration, plating and harvest steps.

The digestion conditions (e.g., size of minced tissue pieces, digestion time, enzyme potency, temperature, stirring speed, tissue/buffer ratio) may influence the speed and amount to which an individual tissue will break down from an intact whole tissue to its individual cellular pieces or components. There is a “sweet spot” between not enough digestion and too much digestion. This “sweet spot” relates to the ratio of single cells to aggregates, as discussed above. According to the present invention, a process is provided which produces a sufficient release of intact follicles from the surrounding connective tissue and blood vessels with an average follicle size that retains the basic architecture of the adjoining epithelial cells and an overall particle size of the tissue breakdown products that is amenable to optimal freezing characteristics (e.g., 20-100 microns in diameter). A spectrum or range of small and large cellular particles may be generated at different rates and proportions depending on the flushing steps, the size, and the digestion conditions listed above.

The particle or aggregate sizes of the digested tissue may be further controlled by filtering the digested tissue through filter(s) that restrict the larger particles from passing through to the final product. During the plating step, retention of the optimal cellular epithelial phenotypes while controlling (but not eliminating) non-epithelial cell types may be an important step for retaining the optimal functional characteristics of the epithelial cells in vitro

Flushing involves cannulating the vessels to the organ and injecting or pumping a fluid through it to remove as much residual blood from the tissue/vessels; blood and its byproducts may be detrimental to cell stability and health. Flushing also stabilizes the tissue and cells while chilling them to reduce their metabolic functions and placing them in a state of suspended animation for transport.

Tissue-culture treated plasticware (flasks) may be used for the growth phase prior to harvesting and freezing to make the release of the cells during harvest occur more easily. A surface coating may allow more attachment of some or all cell types, but may also influence the recovery process in a detrimental way.

A key to successful shipping and transport of sensitive tissues like thyroid is achieving a proper level of suspended animation and temperature, for example, typically in ice-cold or supercooled solutions, during or immediately after recovery from a donor, which minimizes the natural deterioration of tissues and cells in the absence of blood flow, nutrition and oxygenation.

The present invention further comprises a method for preparing a thyrocyte culture. The thyrocyte culture preparation method comprises (a) seeding thyrocytes onto a surface in a culture vessel, wherein the thyrocytes have been isolated from one or more thyroid tissues and cryopreserved; and (b) culturing the thyrocytes in a suitable culture medium, whereby a thyrocyte culture comprising cultured thyrocytes is prepared. The thyrocytes may be seeded at a density of about 1-10×10⁴, for example, 7×10⁴, viable cells/cm², or about 0.1-1,000×10³, 1-1,000×10³, 1-500×10³, 1-100×10³, 1-50×10³ cells per 0.32 cm² in a 96-well plate such that microtissues form by 14 days that are 30-200 microns in diameter in the presence of TSH. According to an embodiment, the seeding density (viable cells) may be 5000-15,000 viable cells per cm² as enumerated using Trypan Blue on a Nexcellom cellometer. The seeding density may be from 2,500-25,000, or from 3,000-20,000, or from 5,000-15,000 viable cells per cm² as enumerated using Trypan Blue on a Nexcellom cellometer. This seeding density may enable microtissue formation of 30-200 uM diameter and secretion of thyroid hormones TG, T3, and T4, as discussed below. According to an embodiment, the thyrocytes that are seeded may comprise about 70% epithelial cells and about 5-30% fibroblast cells. The fibroblast cells may be CD90+. Regarding the CD90+ fibroblasts, it is important to understand that the culture of EpCAM+ epithelial cells in vitro may lead to the phenomenon of epithelial-mesenchymal transition, whereby epithelial cells will begin to concomitantly express markers associated with mesenchymal/stromal cells (such as CD90). Thus, it may be possible that the %-positive CD90 cells may exceed 30%; however, the percent of CD90+ cells that do not concomitantly express EpCAM may be from 5% to 30%.

According to the thyrocyte culture preparation method, the suitable culture medium may comprise one or more supplements selected from the group consisting of charcoal-stripped fetal bovine serum (csFBS), penicillin-streptomycin (Pen-Strep), somatostatin (SMSTN), hydrocortisone (HDCN), growth hormone (GH), apo-Transferrin (APOT), sodium iodide (NaI), sodium selenite (NaSel), L-Glutathione Reduced (GLUT), α-Tocopherol (TOCO), DL-α-Tocopherol acetate (TOCAC), insulin (INS), sodium bicarbonate, and a combination thereof.

The thyrocytes in a culture may be treated with an activator to stimulate microtissue formation and thyroxine hormone production by the thyrocytes. For example, the cultured thyrocytes may be treated with from 0.06 to 3.00 mIU/mL, or 0.07 to 2.5 mIU/mL, or 0.08 to 2.0 mIU/mL, or 0.09 to 1.5 mIU/mL, or from 0.1 to 0.7 mIU/mL, or from 0.2 to 0.5 mIU/mL, or 0.3 mIU/mL TSH on days 2-14 to stimulate the thyrocytes for microtissue formation and thyroxine hormone production. Short treatment on days 2-4 or 2-5 may be sufficient to activate the thyrocytes for thyroxine hormone synthesis.

FBS or charcoal stripped FBS may optionally be used for quality control (QC) and/or functionality studies (e.g., TG/T4). The T4 level in the presence of charcoal stripped FBS may be under the detection level in T4 ELISA, providing a desired dynamic range for thyroid disrupting compound screening.

The thyrocyte culture preparation method may further comprise adding to the growth medium a modifier of thyrocyte stimulating hormone (TSH), a stimulatory thyrotropin receptor antibody, a thyroid stimulating hormone receptor agonist, or a combination thereof. The modifier may be a TSH activator or inhibitor. The TSH may be human TSH (hTSH) or bovine TSH (bTSH). The stimulatory thyrotropin receptor antibody may be M22 or KI-18. M22 is an antibody based TSHR activator isolated from individuals with hyperthyroidism and activates TSHR and leads to T4 production. The thyroid stimulating hormone receptor agonist may be ML-109, or may be K1-70. The thyroid stimulatory or blocking antibodies may be to one or more receptors or pathway intermediates involved in the synthesis of TG, T3, or T4. According to an embodiment the size of the microtissues formed when TSH-stimulated at 14 days of culture on a media such as, but not limited to Matrigel may track with the TG and T4 production. For example, the size of the microtissues formed when TSH-stimulated at 1, 2, 3, 4, 5, 6, 7, 8, 9, 10, 11, 12, 13, 14, 15, 16, 17, 18, 19, 20, 21, 22, 23, 24, 25, 26, 27, or 28 days of culture on a suitable media such as but not limited to Matrigel may track with the TG and T4 production. According to an embodiment, the thyrocyte microtissues may secrete T4 in 3D microtissue culture when exposed to TSH in a range of from about 3-200 ng/ml/48 hrs when wells are seeded with about 5000-30000 cells per well, having been enumerated by Trypan Blue and cellometer.

In the absence of TSH, such as bTSH, the cultured thyrocytes may form smaller microtissues and lack the functionality of the thyroid follicular cells. bTSH may play a role in microtissue formation within an optimal size range and functionality. bTSH may be substituted with M22 to increase the physiologic relevance for disease modeling.

A stimulator such as TSH, bTSH or M22 may be introduced to a thyrocyte culture starting on day 2 in fresh media and then every 2-3 days. Microtissues may be formed by 5-7 days post-plating, and ready for thyroid disrupting chemical (TDC) screening.

The activator or modifier may be introduced to the thyrocyte culture as surrogates for TSH or cellular architecture stimulants such as co-culture, like the triculture model. The activator or modifier may be incorporated into a natural or synthetic hydrogel, such as HuBiogel, or embedded in synthetic or native ECM/scaffolds. Feeder cells and/or hydrogels may be used to support the 3D microtissue architecture, which may be key to forming the colloidal spaces, aligning the subcellular machinery and allowing the production of T4 hormone.

The thyrocyte culture preparation method may further comprise adding one or more supplements to the growth medium. The supplement may be selected from the group consisting of an Epidermal Growth Factor (EGF), a TGFβ inhibitor (e.g., TGFβ type I receptor kinase (ALK5) inhibitor), a fibroblast growth factor (FGF) inhibitor, a Glycogen synthase kinase 3 (GSK-3) inhibitor, thyroid stimulating hormone (TSH), Rho-associated, coiled-coil containing protein kinase (ROCK) inhibitor, and a combination thereof.

According to the thyrocyte culture preparation method, the cultured thyrocytes may comprise a protein selected from the group consisting of cytokeratin 7 (CK7), thyroglobulin (TG), thyroid transcription factor 1 (NKX2-1), epithelial cellular adhesion molecule (EpCAM), thyroid stimulating hormone (TSH) receptor, and thyroperoxidase (TPO) receptor. The cultured thyrocytes may express a gene selected from the group consisting of thyroid stimulating hormone receptor (TSHR), thyroglobulin (TG), thyroperoxidase (TPO), Sodium Iodide Symporter (SLC5A5), Pendrin (SLC26A4), Dual Oxidase 1 (DUOX1), Dual Oxidase 2 (DUOX2), Dual Oxidase Maturation Factor 1 (DUOXA1), and Dual Oxidase Maturation Factor 2 (DUOXA2) genes.

According to the thyrocyte culture preparation method, the culture vessel may be selected from the group consisting of a dish, a flask, a bead, a bioreactor, a chip, a microfluidic chip, an MPS (micro physiological system), a microchip, and a multi-well plate.

According to the thyrocyte culture preparation method, the one or more thyroid tissues may be from one or more donors, for example, from a single donor. Each donor may be a human individual or a non-human vertebrate (e.g., dog, monkey, sheep, cow, horse, mouse, rat, etc.). The human individual may be healthy or suffer from a thyroid disease or disorder. The thyroid disease or disorder may be selected from the group consisting of hyperthyroidism, hypothyroidism, Hashimoto's thyroiditis, Graves' disease, Goiter, thyroid nodules, thyroid tumors, and thyroid cancer.

According to the thyrocyte culture preparation method, the thyrocytes may form microtissues attached to the surface. The resulting culture is a 3D thyrocyte culture. Each of the microtissues may have a hollow space. The microtissues may have a diameter of 50-200 μm. The surface may be coated with an extracellular matrix (ECM). The microtissues may be in contact, directly or indirectly, with the ECM. According to an embodiment, the microtissues may be from about 30-200 μM as measured by image analysis. For example the microtissues may be from 10-250 μM, or from 15-250 μM, or from 20-250 μM, or from 30-250 μM, or from 35-250 μM, or from 40-250 μM, or from 50-250 μM, or from 60-250 μM, or from 70-250 μM, or from 80-250 μM, or from 90-250 μM, or from 100-250 μM, or from 15-200 μM, or from 20-150 μM, or from 30-100 μM, or from 35-90 μM, or from 40-75 μM, or from 50-75 μM as measured Image J software (https://imagej.nih.gov/ij/).

The surface may be coated with an extracellular matrix (ECM). The ECM may comprise one or more molecules selected from the group consisting of cell adhesion peptides, proteoglycans, extracellular matrix proteins, glycosaminoglycans, and admixtures of native tissue-derived proteins. The ECM source may be an Animal-Matrigel or Human-HuBiogel (U.S. Pat. No. 7,727,500), HuGentra (U.S. Application Publication No. US 2018/0155678 A1). The surface may be coated with synthetic or naturally-occurring hydrogels that facilitate cell-cell interaction, non-limiting examples include gelatin, agarose, PDMS, Pluronics™, Poly-HEMA, or similar.

The ECM may be produced naturally by cells or derived from an external source used as a coating to potentially aid attachment.

Low adherence plates are neutrally charged, a hydrophilic coating which helps prevent cell attachment. Low adhesion plates: non-tissue-culture treated plastic; plastic treated with anti-fouling coatings to limit cell attachment (Pluronics, hyaluronic acid, polyhema); non-charged tissue culture surfaces; hydrophobic tissue culture surfaces; surfaces with water contact angles less than 30 degrees or more than 80 degrees.

Matrigel or HuBiogel may be applied to wells of a 96 well plate and incubated in CO₂ incubator at 37° C. for 1 hour to solidify/form gel. Thyrocytes may be immediately thawed and plated onto Matrigel coated plates or alternatively, HTPM may be added on top of Matrigel coated wells and stored in incubator overnight for up to 24 hours prior to thawing and seeding thyrocytes. Cryopreserved cells may be removed from LN2 tank, thawed in 37° C. water bath, and then transferred from vial into conical containing human thyrocyte plating medium (HTPM). Cells may be centrifuged and resulting pellet is resuspended in HTPM and counted using trypan blue. Thyrocytes may be seeded on ECM (Matrigel) coated plated for plates 3D culture. Thyrocytes are cultured for 2 days in HTPM prior to transitioning to the TSH containing human thyrocyte stimulation medium (HTSM). Following the addition of HTSM, formation of microtissue may be present by day 1, day 2, day 3, day 4, day 5, day 6, day 7, day 8, day 9, day 10, day 11, day 12, day 13, or day 14 in culture.

In one embodiment, the plate may be coated with 50 uL Matrigel, which is then polymerized at 37 C for 1 hr. Equilibrium of the ECM with media may be reached overnight. Cryopreserved thyrocytes may be thawed at 37 C temperature-controlled water bath and resuspended in humanized medium (h7H). The resuspended thyrocytes may be seeded at an optimal seeding density and cultured in h7H medium. Bovine, fetal bovine, or human TSH treatment may start on day 2. Recombinant forms of TSH may also be used. TSH treatment may be at from 0.06 to 3.0 mIU/mL, or from 0.1 to 2.5 mIU/mL, or from 0.1 to 2.0 mIU/mL, or from 0.1 to 1.5 mIU/mL, or from 0.1 to 1.0 mIU/mL, or from 0.1 to 0.7 mIU/mL, or from 0.2 to 0.5 mIU/mL, or 0.3 mIU/mL may be applied on days 2-14 with a control group not receiving the bovine TSH treatment. The media may be refreshed every 2-3 days. The media may be collected on Day 14 to determine T4 levels by ELISA or Mass Spec.

The 3D microtissue and cellular architecture of the thyrocytes, for example, colloidal space and follicular structure, may be important for thyroid hormone production. There may be a direct correlation between microtissue size, follicular architecture, colloidal space and efficiency of thyroid hormone production.

The 3D thyrocyte culture preparation method may further comprise producing a hormone selected from the group consisting of thyroxine (T4), 3,5,30-triiodothyronine (T3), monoiodothyronine (MIT) and diiodothyronine (DIT).

The 3D thyrocyte culture preparation method may further comprise exposing the thyrocytes to an agent. The method may further comprise (a) quantifying T4 production in the thyrocyte culture before the exposing; and (b) quantifying T4 production in the thyrocyte culture after the exposing, wherein a change in T4 production after the exposing as compared with that before the exposing indicates that the agent is a modifier of T4 production. The change may be statistically significant, or three times of the baseline median absolute deviation (3×BMAD).

According to the 3D thyrocyte culture preparation method, the agent may be selected from the group consisting of a chemical compound (e.g., thyroid disrupting chemical (TDC)), a biological molecule, small molecules, drugs, antibodies or fragments thereof, purified or synthetic proteins, antibody-drug-conjugates, food additives, cosmetic additives, macro- or micro-environmental stimuli such as physical forces, oxygen tension, and the like, and a combination thereof. The agent may be a therapeutic agent for treating a thyroid disease or disorder. The agent may be a preventive agent for preventing a thyroid disease or disorder. The thyroid disease or disorder may be selected from the group consisting of hyperthyroidism, hypothyroidism, Hashimoto's thyroiditis, Graves' disease, Goiter, thyroid nodules, thyroid tumors, and thyroid cancer.

In one embodiment, a thyroid disrupting chemical (TDC) may be screened using a 3D thyrocyte culture. Eight doses of a test compound may start on Day 9. The thyrocyte culture may be refreshed with media containing the test compound on Day 12. The supernatant of the thyrocytes may be collected on Day 14 for T4 analysis by ELISA.

The time of starting the test compound is not particularly limited and may be adjusted according to expected or actual results from the testing. According to some embodiments, the test compound may be started on day 1, 2, 3, 4, 5, 6, 7, 8, 9, 10, 11, 12, 13 or 14. The culture time is also not particularly limited and the cultures may be taken beyond 14 days, such as 15, 16, 17, 18, 19, 20, 21, 22, 23, 24, 25, 26, 27, or 28 days. The test compound may likewise be started at a time after 14 days, The number of doses is also not particularly limited that number of does may be 1, 2, 3, 4, 5, 6, 7, 8, 9, 10, 11, 12, 13, 14 or more. The dose may be administered daily, twice a day or three times a day. A single dose may be given, or the dose(s) may be given 1 or 2 or more days apart.

To shorten the assay period, the treatment may start on day 5 and end on Day 10. Serial dilution may be prepared in a solvent (e.g., DMSO, PBS) and added into the media (1:1000 dilutions).

In another embodiment, TDC screening in a 3D thyrocyte culture may comprise adding a test compound on Day 5; Refreshing media with the compound on Days 7-8; and collecting the supernatant on Day 10 for the T4 analysis by ELISA or mass spectrometry

According to the thyrocyte culture preparation method, the cultured thyrocytes may form a monolayer in direct contact with the surface. The resulting culture is a 2D thyrocyte culture. The surface may be coated with fibronectin or poly-lysine. The surface may not be coated with an extracellular matrix (ECM).

The 2D thyrocyte culture preparation method may further comprise exposing the thyrocytes to an agent. The method may further comprise (a) quantifying thyroglobulin (TG) production in the thyrocyte culture before the exposing; and (b) quantifying TG production in the thyrocyte culture after the exposing, wherein a change in TG production after exposing as compared with that before the exposing indicates that the agent is a modifier of TG production. The change may be statistically significant, or three times of the baseline median absolute deviation (3×BMAD).

According to the 2D thyrocyte culture preparation method, the agent may be selected from the group consisting of a chemical compound (e.g., thyroid disrupting chemical (TDC)), a biological molecule, and a combination thereof. The agent may be a therapeutic agent for treating a thyroid disease or disorder. The agent may be a therapeutic agent for preventing a thyroid disease or disorder. The thyroid disease or disorder may be selected from the group consisting of hyperthyroidism, hypothyroidism, Hashimoto's thyroiditis, Graves' disease, Goiter, thyroid nodules, thyroid tumors, and thyroid cancer.

The cryopreserved thyrocytes may be seeded on ECM (Matrigel) coated plate for making a 3D culture or non-ECM tissue culture plastic (TCP) plate for making a 2D culture. The thyrocytes may be cultured for 2 days in HTPM prior to evaluating a biomarker in the 2D culture using IFC for CK7 and/or TG or continuing a 3D culture in TSH-containing human thyrocyte stimulation medium (HTSM). Medium from a 3D culture may be collected on day 14 of culture for evaluation of T4 production.

For each 3D thyrocyte culture preparation method, a 3D thyrocyte culture prepared according to the method is provided.

For each 2D thyrocyte culture preparation method, a 2D thyrocyte culture prepared according to the method is provided.

In one embodiment, a product kit may be provided to a customer to test a chemical compound to assess its safety or test a therapeutic agent for its treatment efficacy. The kit comprises a thyrocyte component, a set of application media with no or trace amount of background TSH or other thyroid hormone level, optionally a tissue culture device prepared with an animal or human ECM (all human system), and an instruction of use (IFU) with recommended conditions.

In one embodiment, a thyrocyte culture system may be provided to a customer to test a chemical compound to assess its safety or test a therapeutic agent for its treatment efficacy. The culture system comprises a thyrocyte component, the thyrocyte is cultured on a device coated with an animal or human ECM, and the thyrocyte is cultured in a culture media. The culture media may have no or only a trace amount of background TSH or other thyroid hormone level. Such a culture media may offer a higher signal to noise ratio.

In one embodiment, a thyrocyte culture system may be provided to a customer to test a therapeutic agent for its treatment efficacy. The culture system comprises a thyrocyte component, the thyrocyte is cultured on a device coated with an animal or human ECM, a set of application media. The thyrocyte may be obtained from a patient with a thyroid disease, and in a culture media with no or trace amount of background TSH or other thyroid hormone level.

In an embodiment, a thyrocyte product and thyrocyte system may be used for in vitro assessment of safety, toxicity, carcinogenicity, tumorigenicity, endocrine disruption, thyroid hormone disruption. Non-limiting examples of test articles include: small molecules, proteins, mammalian cells, bacterial cells, antibody-drug conjugates (ADCs), antibodies, synthetic or naturally-occurring proteins, environmental or microenviromental cues such as oxygen tension, stress/strain, pressure, shear, light/dark cycle.

According to an embodiment, the thyrocyte product may be used for treatment of thyroid dysregulation by providing thyrocytes to a mammal with dysregulated thyroid function. The thyrocytes may be allogeneic, autologous, or xenogeneic. The thyrocytes may be delivered in vivo, to at least one of the following: orthotopic site, ectopic site, contained within an implantable device that supports regulated production and release of thyroid hormones. In an embodiment, the thyrocytes may be delivered ex vivo via a wearable device that enables regulated production, release, and delivery of thyroid hormones.

As used herein, the term “about” modifying, for example, the dimensions, volumes, quantity of an ingredient in a composition, concentrations, process temperature, process time, yields, flow rates, pressures, and like values, and ranges thereof, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of, for example, a composition, formulation, or cell culture with a particular initial concentration or mixture, and amounts that differ due to mixing or processing a composition or formulation with a particular initial concentration or mixture. Whether modified by the term “about” the claims appended hereto include equivalents to these quantities. The term “about” further may refer to a range of values that are similar to the stated reference value. In certain embodiments, the term “about” refers to a range of values that fall within 10, 9, 8, 7, 6, 5, 4, 3, 2, 1 percent or less of the stated reference value.

### EXAMPLES

### Example 1. Thyrocyte Isolation

A human thyroid tissue was recovered from a donor and kept on ice. Excess surrounding adipose or connective tissue was removed from the recovered thyroid tissue. The thyroid tissue was cut into small pieces and then added to a digestion cocktail containing collagenase IA (1.25 mg/ml) and collagenase IV (2.5 mg/ml), which was pre-warmed to 37° C. Note: each lot of these enzymes has a specific activity as expressed in International Units (IU)/mg. It should be noted that concentrations in other examples or use cases may vary slightly on a mg/ml basis reflecting adjustment to IU/mg. The tissue was incubated in the digestion cocktail with constant stirring a on stir plate for up to 1 hour at 37° C. and 5% CO₂. Starting at 30 minutes, the tissue digestion was monitored every 10 minutes up to 1 hour for the presence of follicular aggregates and single cells, an indicator of complete tissue digestion. Digestion was quenched in a h7H medium and the cell suspension was transferred to conical tubes. The tubes were centrifuged at 300 g for 10 min followed by a wash with warm HBSS and recentrifugation. The resulting pellet was resuspended in the h7H medium. Cell count and viability were determined using trypan blue exclusion on a hemacytometer. The resuspended cells were seeded onto a culture surface in flasks at 7×10⁴ viable cells/cm2 in the h7H medium. The h7H medium was changed daily until the surface in the flasks reached 90-100% confluence in 4-6 days.

### Example 2. Thyrocyte Cryopreservation

After reaching desired confluency, the cultured thyrocytes were rinsed three times with warm DPBS (−/−) and dissociated from the culture surface using TrypLE Select at 37° C. and 5% CO₂. The dissociation of the cultured thyrocytes was then monitored every 5 minutes until ~95% of the cultured thyrocytes were dissociated from the culture surface and became free moving single cells and small aggregates (<10 cells). The TrypLE was quenched by adding a h7H medium. The thyrocyte suspension was transferred to conical tubes and centrifuged at 300 g for 10 min. The resulting pellet was resuspended in a NG5A cryopreservation medium. Cell count and viability of the suspended thyrocytes were determined using trypan blue exclusion on a hemacytometer. The NG5A cryopreservation medium was added to give 1.2×10⁶ cells/ml. An aliquot of 1 ml thyrocyte suspension was added to a vial and cryopreserved using a controlled rate freezer. The frozen vials were transferred to an LN₂ storage dewar.

### Example 3. Preparation of Media for Isolation, Thawing, and Culture of Thyrocytes

Supplements (Table 1) were prepared for a h7H medium, which was used for isolation of thyrocytes.

TABLE 1
Supplements for h7H medium

| | Stock Solution | | Storage | | Volume | | Final Media | |
|---|---|---|---|---|---|---|---|---|
| Supplement | Concentration | | Conditions | | to Add | | Concentration | |
| Fetal bovine serum [FBS] | 100% | | −20° | C. | 100 | mL | 10% | |
| Penicillin-Streptomycin [Pen-Strep] | 10X | | −20° | C. | 10 | mL | 1X | |
| Somatostatin [SMSTN] | 50 | μg/mL | −20° | C. | 1 | μL | 50 | ng/L |
| Hydrocortisone [HDCN] | 1 mg/mL | | −20° | C. | 8.3 | μL | 8.3 μg/L | |
| | [2.76 mM] | | | | | | [23 nM] | |
| Growth hormone [GH] | 10 | μg/mL | −20° | C. | 20 | μL | 0.2 | μg/L |
| apo-Transferrin [APOT] | 1 | mg/mL | −20° | C. | 5 | mL | 5 | mg/L |
| Sodium iodide [NaI] | 1 | mg/mL | −20° | C. | 10 | μL | 10 | μg/L |
| Sodium selenite [NaSel] | 25 | mg/mL | −20° | C. | 3 | μL | 75 | μg/L |
| L-Glutathione Reduced [GLUT] | 25 | mg/mL | −20° | C. | 8 | μL | 0.2 | mg/L |
| α-Tocopherol [TOCO] | 50 | mg/mL | 4° | C. | 10 | μL | 0.5 | mg/L |
| DL-α-Tocopherol acetate [TOCAC] | 50 | mg/mL | 4° | C. | 10 | μL | 0.5 | mg/L |
| Insulin [INS] | 9.5-11.5 | mg/mL [symbol] | 4° | C. | Variable [symbol] | | 20.7 | IU/L |
| Bovine thyroid stimulating hormone [bTSH] | 0.5 | IU/mL | −20° | C. | 80 | μL | 40 | mIU/L |

[symbol]

indicates data missing or illegible when filed

Supplements (Table 2) were prepared for a human thyrocyte plating medium (HTPM), which was used for plating thyrocytes for quality control (QC) testing.

TABLE 2
Supplements for HTPM

| | Stock Solution | | Storage | | Volume | | Final Media | |
|---|---|---|---|---|---|---|---|---|
| Supplement | Concentration | | Conditions | | to Add | | Concentration | |
| Charcoal-stripped fetal bovine serum [c [symbol] FBS] | 100% | | −20° | C. | 100 | mL | 10% | |
| Penicillin-Streptomycin [Pen-Strep] | 10X | | −20° | C. | 10 | mL | 1X | |
| Somatostatin [SMSTN] | 50 | μg/mL | −20° | C. | 1 | μL | [symbol] 0 | ng/L |
| Hydrocortisone [HDCN] | 1 mg/mL | | −20° | C. | 8.3 | μL | 8.3 μg/L | |
| | [2.76 mM] | | | | | | [23 nM] | |
| Growth hormone [GH] | 10 | μg/mL | −20° | C. | 20 | μL | 0.2 | μg/L |
| apo-Transferrin [APOT] | 1 | mg/mL | −20° | C. | 5 | mL | 5 | mg/L |
| Sodium iodide [NaI] | 1 | mg/mL | −20° | C. | 10 | μL | 10 | μg/L |
| Sodium selenite [NaSel] | 25 | mg/mL | −20° | C. | 3 | μL | 75 | μg/L |
| L-Glutathione Reduced [GLUT] | 25 | mg/mL | −20° | C. | 8 | μL | 0.2 | mg/L |
| α-Tocopherol [TOCO] | 50 | mg/mL | 4° | C. | 10 | μL | 0.5 | mg/L |
| DL-α-Tocopherol acetate [TOCAC] | 50 | mg/mL | 4° | C. | 10 | μL | 0.5 | mg/L |

| | | | | | | | |
|---|---|---|---|---|---|---|---|
| Insulin [INS] | 9.5-11.5 | mg/mL [symbol] | 4° | C. | Variable [symbol] | 20.7 | IU/L |

[symbol]

indicates data missing or illegible when filed

The human thyrocyte stimulation medium (HTSM) is the HTPM supplemented with bTSH at 0.3 mIU/ml during QC testing.

### Example 4. Thawing of Cryopreserved Human Thyrocytes

Vials of cryopreserved thyrocytes were removed from LN2 storage and quickly transferred to a water bath at 37° C. to thaw the cryopreserved thyrocytes for 90 seconds or until thawed. The thawed thyrocytes were resuspended and added dropwise to 14 mL HTPM using a P1000 pipet, and the resulting mixture was then centrifuged at 200 g for 5 minutes. The resulting pellet was resuspended in HTPM. Cell count and viability were then determined using trypan blue exclusion with Cellometer.

### Example 5. Plating and Culturing Human Thyrocytes

Working on ice, wells of a clear 96 well plate were coated with 50 μl of Matrigel at a concentration of ~10 mg/ml using chilled pipet tips. Coated plates were incubated at 37° C. and 5% CO₂ for 1 hour. Thawed cryopreserved human thyrocytes were resuspended in HTPM to achieve a density of 7.5×10⁴ cells/ml. The thyrocyte suspension was added to a sterile reservoir and 100 μl of the thyrocyte suspension was added to the Matrigel coated wells for making a 3D culture and wells of tissue culture treated black walled clear bottom 96 well plate for making a 2D culture using a multichannel pipet. For the 2D culture, the cultured thyrocytes were imaged and processed for ICC on day 2. For the 3D culture, a TSH supplemented medium was added to designated (treated) wells starting on day 2. Microtissues were observed in the 3D culture and medium changes were performed on days 2, 5, 7, 9, 12, and 14. The medium was collected on day 7 for TG ELISA and on day 14 for T₄ ELISA.

### Example 6. Purity and ID of Human Thyrocytes in 2D Cultures

1× Perm/Wash buffer was prepared by diluting 10× Perm/Wash Buffer in deionized water. The medium was removed from the black walled 96 well plate and 100 μl of a BD Fix/Perm solution was added to each well. The plate was incubated at 4° C. for 30 minutes and then the wells were washed twice with 200 μl of 1× Perm/Wash Buffer. Antibodies for TG (Alexa 488 conjugated) and CK7 are diluted 1:100 in 1× Perm/Wash Buffer and 100 μl of prepared antibodies are added to each designated well. Following overnight (16-24 hrs) incubation at 4° C., wells are washed 3 times with 200 μl of 1× Perm/Wash Buffer. Goat anti-mouse Alexa 488 IgG (H+L) secondary antibody is prepared at 1:500 dilution in 1× Perm/Wash and 100 μl of secondary antibody is added to CK7 stained wells. Wells are washed 3 times with 1× Perm/Wash Buffer and then 100 μl of Fluoromount G with DAPI is added to each well. Wells imaged under fluorescence microscope at 10× magnification.

### Example 7. Thyroglobulin ELISA for Human Thyrocytes

Standards prepared as part of a TG ELISA kit were prepared in Assay Diluent C. On day 7, the medium in a TSH treated or non-treated 2D thyrocyte culture was collected and centrifuged for 10 minutes at 1500 rpm at room temperature. The resulting supernatant was diluted with Assay diluent C at 1:100. 100 μl of each standard and samples were added to wells of a 96 well ELISA plate in duplicate. The plate was incubated overnight for 16-24 hours at 4° C. The wells were washed four times with 300 μl of 1× Wash Buffer using a multichannel pipette and then 100 μl of biotin conjugate diluted in 1× Assay Diluent B was added to each well. Following 1 hour incubation at room temperature with shaking at 300 rpm, the wells were washed again four times and 100 μl of 1× Streptavidin-HRP solution diluted in 1× Assay Diluent B was added to each well. The plate is incubated for an additional 45 minutes at room temperature while shaking at 300 rpm. The wells were then washed again four times and 100 μl 3,3′,5,5′-Tetramethylbenzidine (TMB) substrate was added to each well. The plate was incubated in dark for 30 minutes with shaking at 300 rpm and then 50 μl of a stop solution was added to each well. The plate was read on a microplate reader at 450 nm and 550 nm within 30 minutes after the addition of the stop solution. TG concentrations of the samples were determined from four parameter logistic curve fit and then normalized.

### Example 8. Thyroglobulin T4 Competitive ELISA for Human Thyrocytes

Standards prepared as part of T4 ELISA kit were prepared in 1× Assay Buffer. On day 14, the medium in a TSH treated or non-treated 3D thyrocyte culture was collected and centrifuged for 10 minutes at 1500 rpm at room temperature. 10 μl of each standard and sample, 25 μl of Thyroxine conjugate, and 25 μl of Thyroxine antibody were added to each well in a 96 well ELISA plate in duplicate. The plate was covered with plate sealer and incubated for 1 hour at room temperature with shaking at 300 rpm. The wells were washed four times with 300 μl of 1× Wash Buffer using a multichannel pipette and then 100 μl of a 3,3′,5,5′-Tetramethylbenzidine (TMB) substrate was added to each well. The plate was incubated for 30 minutes at room temperature without shaking and 50 μl of stop solution was added to each well. The plate was read on a microplate reader at 450 nm and 570 nm within 30 minutes. T4 concentrations of the samples were determined from four parameter logistic curve fit and then normalized.

### Example 9. 2D Thyrocyte Cultures

### Materials and Methods

### Assessment of Activators

Cryopreserved thyrocytes were thawed in a water bath at 37° C. The thawed cells were resuspended in humanized medium (h7H). The resuspended cells were seeded at 10,000 cells per 0.32 cm² for protein analysis or 40,000 cells per 0.32 cm² for mRNA gene expression analysis. The cells were cultured in the h7H medium, and treated with a TSHR activator, which may be a small molecule, antibody-based activator or naïve TSHR activator, on Days 2-7. The cells were then harvested on Day 7. RNA was isolated from the harvested cells for mRNA expression assays. Supernatant of the harvested cells was used for TG protein analysis.

### Preventive Drug Screening

Cryopreserved thyrocytes were thawed in a water bath at 37° C. The thawed cells were resuspended in humanized medium (h7H). The resuspended cells were seeded at 10,000 cells per 0.32 cm² for protein analysis or 40,000 cells per 0.32 cm² for mRNA gene expression analysis. The cells were cultured in the h7H medium, and treated with a TSHR activator, which may be a small molecule, antibody-based activator or naïve TSHR activator, on Days 7-12. The cells were treated with a test agent on Days 7-12. The cells were then harvested on Day 12. RNA was isolated from the harvested cells for mRNA expression assays. Supernatant of the harvested cells was used for TG protein analysis.

### Therapeutic Drug Screening

Cryopreserved thyrocytes were thawed in a water bath at 37° C. The thawed cells were resuspended in humanized medium (h7H). The resuspended cells were seeded at 10,000 cells per 0.32 cm² for protein analysis or 40,000 cells per 0.32 cm² for mRNA gene expression analysis. The cells were cultured in the h7H medium, and treated with a TSHR activator, which may be a small molecule, antibody-based activator or naïve TSHR activator, on Days 2-12. The cells were treated with a test agent on Days 7-12. The cells were then harvested on Day 12. RNA was isolated from the harvested cells for mRNA expression assays. Supernatant of the harvested cells was used for TG protein analysis.

This study showed treatment with activator from days 2-7/8 and that TG was selected as reporter gene based its dynamic range. The study was extended with M22 IgG (+) with the following groupings”
Group 1: No treatment with the activator
Group 2: Treatment with the activator on Day 2-7
Group 3: Treatment with the activator Day 2-14
Group 4: Treatment with the activator Day 2-14+DMSO on Days 7-14
Group 5: Treatment with the activator Day 2-14+K1-70 IgG (+) on Days 7-14

Results demonstrated that TSHR could be activated by various activators, including TSH or antibodies that interact with TSHR.

### Example 10. 3D Thyrocyte Culture

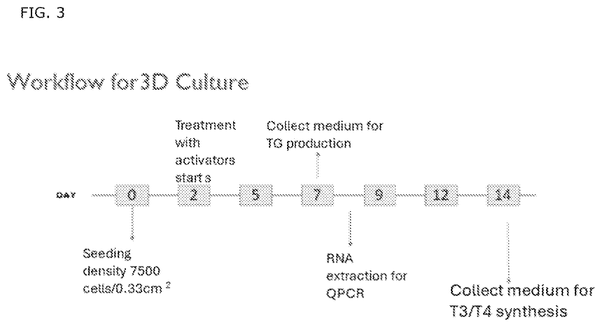

The workflow for 3D Culture for T4 synthesis is shown in FIG. 3. The procedures for Example 10 are as follows:
Prepare the Plate by adding 50 μl of 10 mg/ml of Matrigel, then polymerize the Matrigel at 37° C. for 1 hr.
Thaw the thyrocytes in 37° C. water bath.
Resuspend the cells in humanized medium (h7H).
Seed the cells at the optimal seeding densities at 15,000-2,500 cells per 0.32 cm2.
Culture the cells in h7H medium.
Exchange the medium on Day 2, 5, 7, 9 and 12.
   Start stimulating the cells with activators (e.g., bSH, hTSH, M22, K1-18, ML-109) on Day 2
Treatment with the activator is between Day 2-14.
Collect supernatant on Day 7 to determine TG production (protein) by ELISA (alternatively, it can be as early as Day 2 or late as day 14).
Collect supernatant on Day 5, 7, 9, 12 and 14 to determine T3/T4 production (protein) by ELISA and LC-MS/MS.

The results for Example 10 demonstrated that TSH promotes 3D microtissue formation, TG secretion and T3/T4 synthesis. All qualified lots maintained native thyroid functions including TG secretion and T4 synthesis when the cells were activated with bTSH. Thyrocytes synthesized T4 in 3D cultures when stimulated with bTSH, but not 2D culture. The results showed that optimal seeding density can be lowered to 3.75E+03 cells per well for LC_MS/MS (7.5E+03 cells per well for ELISA) and that optimal 3D thyroid microtissues size occurred at seeding densities from 7.5E+03 to 2.5E+03 cells per well. The data form ELISA provided that screening of TDCs can be started as early as Day 5 compared to Day 10.

For mRNA Profiling, these procedures were followed:
Prepare the Plate adding 50 uL of 10 mg/ml of Matrigel, then polymerize the Matrigel at 37° C. for 1 hr.
Thaw the thyrocytes in 37° C. water bath.
Resuspend the cells in humanized medium (h7H).
Seed the cells at the seeding density, 7,500 cells per 0.32 cm2 (6 wells per condition).
Culture the cells in h7H medium
Exchange the medium on Day 2, 5, 7.
   Start stimulating the cells with activators (e.g., bTSH, hTSH, M22, K!-18, ML-109) on Day 2
Treatment with the activator is between Day 2-8.
Harvest the cells on day 8 and isolate RNA.

For TDC Screening, these procedures were followed:
Prepare the Plate by adding 50 μL of 10 mg/ml of Matrigel, then polymerize the Matrigel at 37° C. for 1 hr.
Thaw the thyrocytes in 37° C. water bath.
Resuspend the cells in humanized medium (h7H).
Seed the cells at the optimal seeding density, 7,500 cells per 0.32 cm2.
Culture the cells in h7H medium.
Exchange the medium on Day 2, 5, 7, 9 and 12.
   Start stimulating the cells with activators (e.g., bTSH, hTSH, M22, K!-18, ML-109) on Day 2
Treatment with the activator is between Day 2-14
Compound treatment starts on Day 9.
   8 doses: Concentrations (10⁻¹⁰ to 10⁻⁴M)
      Serial dilutions were made in DMSO (Dilution factor 1:1000)
      Compounds were added to the media
   Day 12: Refresh media with the compound
   Day 14: Collect the supernatant for the T4 analysis by ELISA and LC-MS/MS.

### Alternative:

Compound treatment can also start as early as day 5.
~Day 7: Refresh media with the compound
~Day 10: Collect the supernatant for the T4 analysis by ELISA and LC-MS/MS.

The results for Example 10 established that T3/T4 synthesis was inhibited with TPO inhibitors; methimazole and 6-propyl-2-thiouracil.

### Example 11. 3D Culture TDC Screening

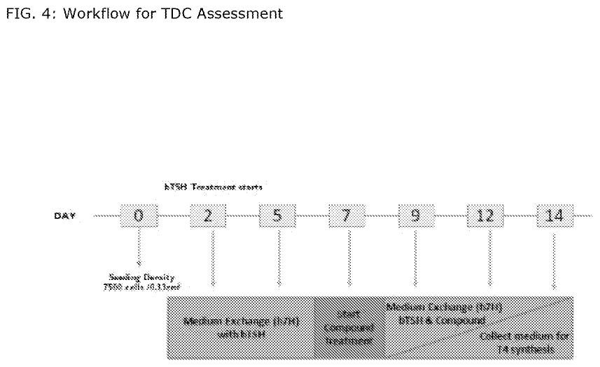

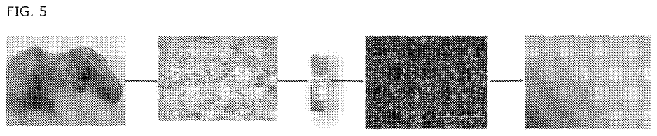

The workflow for 3D Culture TDC screening is shown in FIG. 4. The procedures for Example 11 are as follows:
Thaw the thyrocytes in 37° C. water bath.
Resuspend the cells in humanized medium (h7H).
Seed the cells at the optimal seeding density, 7,500 cells per 0.32 cm2.
Culture the cells in h7H medium.
Treatment with the activator is between Day 2-14.
Compound treatment starts on Day 9.
   8 doses: Concentrations (10⁻¹⁰ to 10⁻⁴M)
      Serial dilutions are made in DMSO (Dilution factor 1:1000)
      Compounds were added to the media.
Day 12: Refresh media with the compound.
Day 14: Collect the supernatant for the T4 analysis by ELISA.

### Example 12

Methods: Thyroid epithelial cells (passage 1) from healthy adult donors (males and females; ≤55 years of age; body mass index of ≤35) were cryopreserved in a serum-free biopreservation medium. Cryopreserved cells from individual donors (n=3) were thawed in human thyrocyte plating medium (HTPM) and plated in 96-well TC-treated culture plates. The thyrocytes were maintained in an h7H-based culture medium containing a TSHR activator for 8 days. Thyroglobulin (TG) mRNA and protein levels were determined using QPCR and ELISA, respectively.

Results: Results showed that native (bovine and human thyroid stimulating hormone [TSH]) and antibody-based activators (M22 IgG (+) and K1-18 IgG (+)) induced TG mRNA expression levels in a concentration-dependent manner and half-maximal effective concentration (EC50) values were 0.12, 0.384, 0.358 and 1.21 nM, respectively. Inhibition of TG synthesis in cells stimulated with M22 IgG (+) at its EC80 concentration was validated following a 120-hr treatment with the TSHR inhibitor, K1-70 IgG (+) using therapeutic and preventive treatment methods. Half-maximal inhibitory concentrations (IC50) for K1-70 IgG (+) were 3.89 and 6.26 nM for preventive and therapeutic treatment methods, respectively. AITD response was reproduced with antibody-based activators using the primary human thyrocytes.

### Example 13: Cryopreserved Primary Human Thyrocytes for Discovery and Development of Thyroid Disease Treatments

### Materials and Methods

Cell culture of cryopreserved thyrocytes: Thyrocytes from healthy donors were isolated as described in Example 1 and cryopreserved as in Example 2. FIG. 5 shows a schematic representation of PHT (primary human thyrocytes) isolation and quality control in Example 13. The cryopreserved thyrocytes (p1) were thawed in h7H base human thyrocyte plating media (HTPM) and plated in 96-well TC-treated culture plates. Cell cultures were treated with native TSHR activators (bovine and human) or antibody-based activators (M22 IgG (+) and K1-18 IgG (+)) on day 2-8.

The characteristics of the donors are shown in Table 3.

TABLE 3

| Donor ID | Sex | Age | Race | BMI |
|---|---|---|---|---|
| THY2020525 | M | 22 | C | 29.1 |
| THY2024220 | M | 50 | AA | 21.6 |
| THY2211979 | F | 27 | AA | 29.8 |
| THY2215688 | M | 42 | H | 36 |

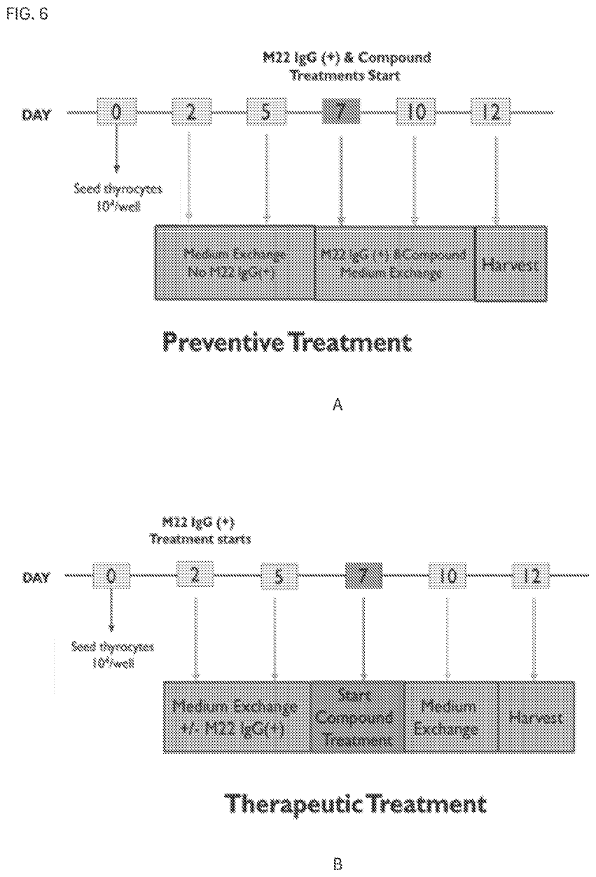

FIG. 6 shows the timeline of preventative (A) and therapeutic (B) treatment of these cell cultures.

Assay analysis: For mRNA analysis, total RNA was isolated using Qiagen RNEasy Mini Kit (Qiagen, 74106) and quantified with NanoDrop 8000 spectrophotometer (ThermoFisher, ND-8000-GL). Isolated total mRNA was reverse transcribed into cDNA using the High-Capacity cDNA Reverse Transcription Kit (Applied Biosystem, 4368813). qPCR analysis was performed using Tagman Universal PCR Mastermix (Applied Biosystem, 4304437) with the following probe (ThermoFisher): Hs00427620_m1 (TBP) and hs00794359_m1 (TG).

TG production at protein level was analyzed using ELISA kits (Invitrogen, EHTG).

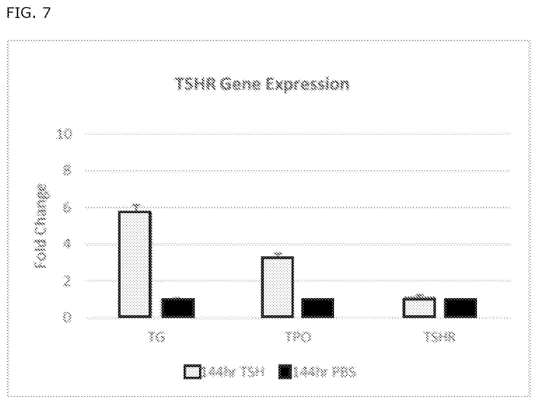

Data analysis: Data was normalized to vehicle controls and plotted using GraphPad Prism. The results are shown in FIGS. 7-11.

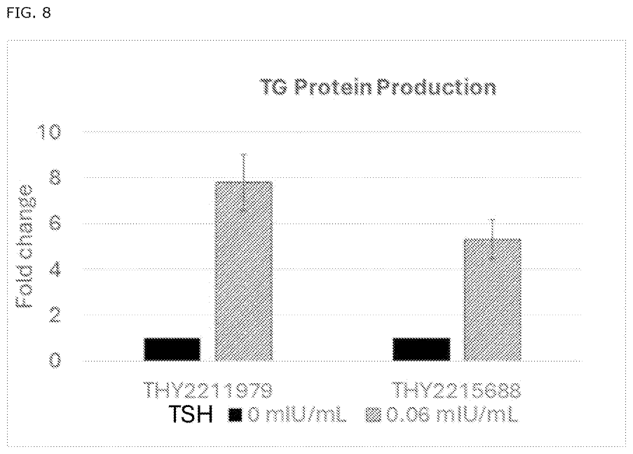

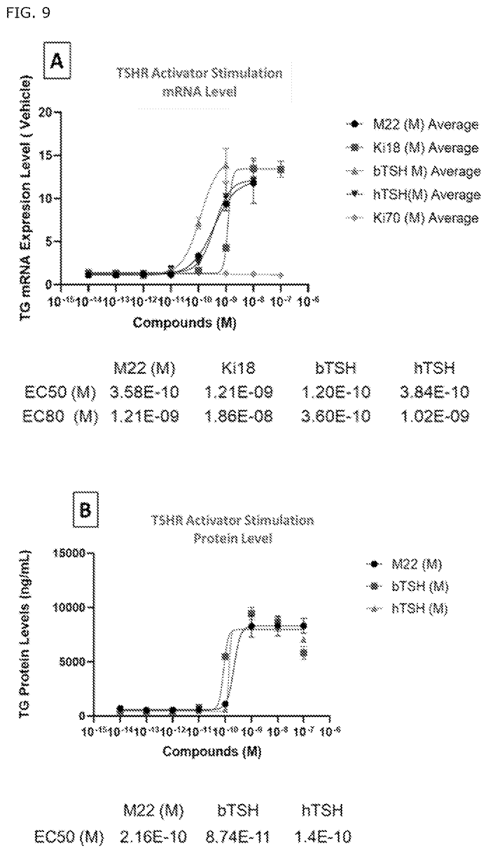

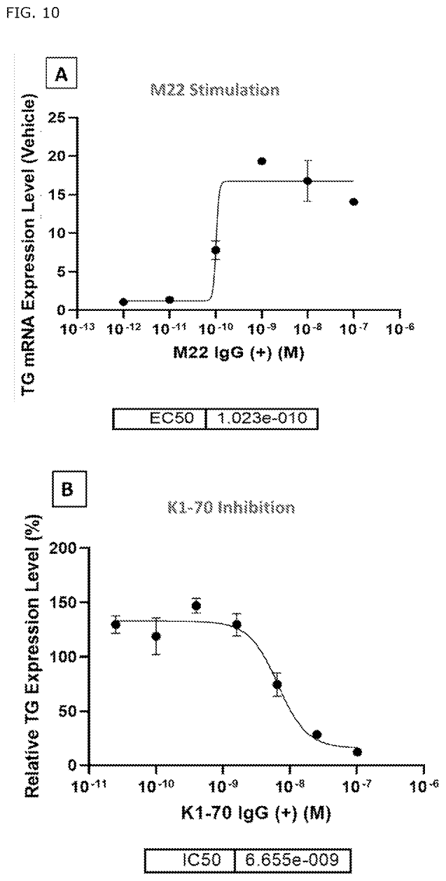

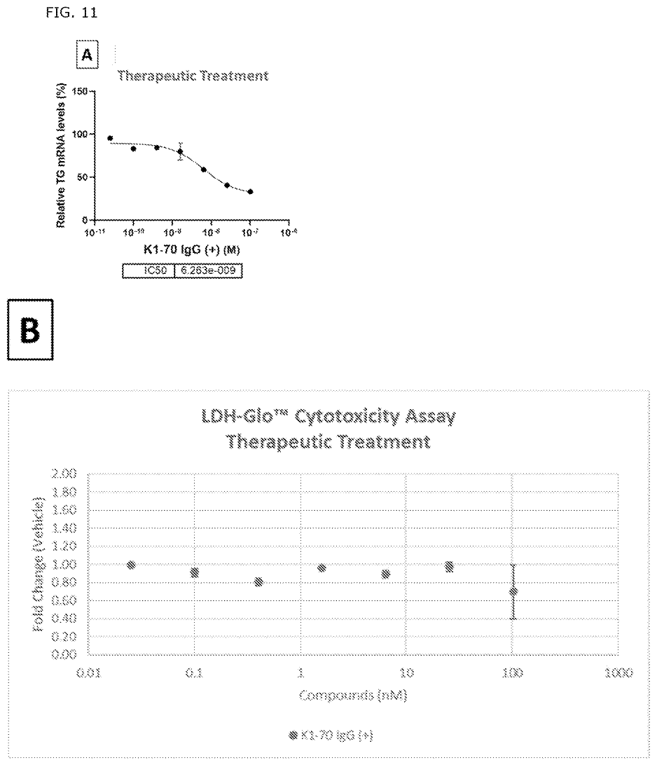

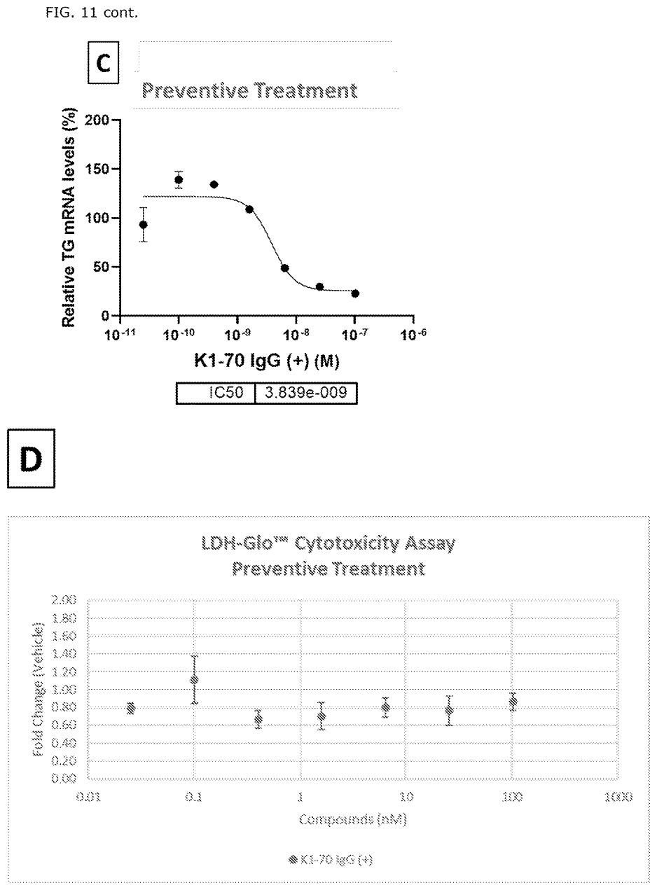

FIG. 7 shows the reporting genes identification as assessed by fold change. The thyrocytes were treated with either PBS or 0.3 ml/mL TSH on days 2-8. FIG. 8 shows TG protein production as validation of additional thyrocyte lots as assessed by fold change. The thyrocytes were treated with 0 or 0.06 mIU/mL of TSH on days 2-8. FIGS. 9A and 9B show TG production in the presence of TSHR activators. Thyrocyte cultures were maintained in media containing either native (bTSH and hTSH) or antibody-based (M22 or Ki18) TSHR activators (day 2-8). TSHR inhibitor Ki-70 was used as negative control. FIG. 9A shows TG production at mRNA and FIG. 9B shows TG production at protein levels. FIGS. 10A and 10B show TG production in a dose-response manner using (A) M22 and (B) K1-70. For K1-70 compound study, cell cultures were stimulated with M22 IgG (+) at EC80 value. FIGS. 11A-D show a comparison of K1-70 treatment conditions. FIGS. 11A and 11B show TG mRNA level production and cytotoxicity effect of K1-70 for therapeutic treatment. FIGS. 11C and 11D show production and cytotoxicity effect of K1-70 for preventative treatment. Cell cultures were stimulated with M22 IgG (+) at the EC80 value.

In conclusion, TG was identified as the reporter gene for thyrocyte model validation with the time of exposure to the activators of 8 days (n=3). TG mRNA and protein levels were upregulated by TSHR activators in a dose-response manner while TSHR inhibitor, K1-70, inhibited TG production. Preventative treatment using K1-70 inhibitor resulted in a better inhibition response compared to therapeutic treatment. Thus, this example demonstrates that these cryopreserved primary human thyrocyte model cultures may be used for high-throughput compound screening.

### Example 14. Diameter of Cell Aggregates

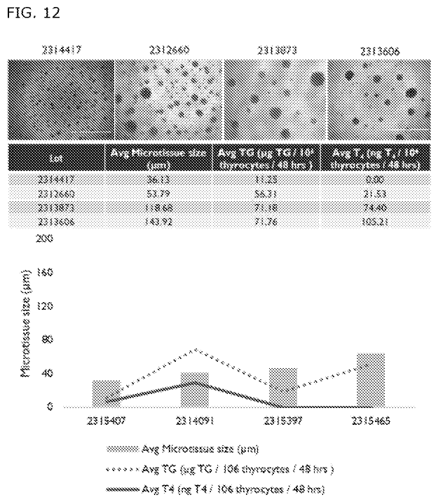

FIG. 12 shows the diameter of cell aggregates (microtissues/cell aggregates) tracks with T4 production. Microtissues that are less than about 30-40 microns in size are poor producers of thyroid hormones, suggesting there is an optimal size range to achieve T4 production. 3D culture systems that facilitate generation and maintenance of cell aggregates within that size range will be advantageous for applications in vitro that require detection of thyroid hormone modulation (stimulation or inhibition), such as TSH exposure or TDC compound exposure. Average microtissue size was calculated by image analysis using Image-J software, in 4 lots of cryopreserved thyrocytes. T4 and TG secretion were quantified by ELISA assay. Interestingly, small microtissue size was associated with low hormone production and larger microtissue size with higher levels of T4 and TG.

### Example 15: Frozen Supplement

A frozen supplement was generated containing Insulin, Somatostatin, Hydrocortisone, Growth Hormone, Apotransferrin, Sodium Iodide, Sodium Selenite, Glutathione, Vitamin E (Alpha-Tocopherol), and DL-alpha Tocopherol Acetate. Freshly prepared media was compared across three thyrocyte lots with respect to yield, viability, 3D microtissue formation, T4 levels and T3 levels. The results are shown in the table below. Note the concordance in performance between the freshly prepared media and the frozen supplement.

| | Passing | | | | Base Media and Frozen | | |
|---|---|---|---|---|---|---|---|
| Specification | Criteria | Fresh Media | | | Supplement | | |
| Cells | At passage 1 | Pass | | | Pass | | |
| Sterility | Negative for | PASS | | | PASS | | |
| | mycoplasma, | | | | | | |
| | bacteria, | | | | | | |
| | fungi, and | | | | | | |
| | yeast | | | | | | |
| Viability and | ≥0.5 × 10⁴ | LOT A | | LOT B | | LOT C | |
| Cell Yield | cells/vial | 2.61 × 10⁶ ± | | 3.37 × 10⁶ ± | | 4.23 × 10⁶ ± | |
| | | 1.79 × 10⁵ | | 4.37 × 10⁵ | | 5.92 × 10⁵ | |
| | | PASS | | PASS | | PASS | |
| | ≥70% viable | 80.04% ± 4.57% | | 82.88% ± 5.41% | | 75.2% ± 5.17% | |
| | post thaw | PASS | | PASS | | PASS | |
| | (trypan | | | | | | |
| | blue) | | | | | | |
| 3D | 3D | PASS | | | PASS | | |
| Microtissue | microtissue | | | | | | |
| | formation by | | | | | | |
| | day 7 | | | | | | |
| T4 levels | ≥3.87 ng | LOT A | LOT B | LOT C | LOT A | LOT B | LOT C |
| | T4/10⁴ | 19.75 ± | 2.12 ± | 27.81 ± | 18.18 ± | 0.00 ± | 28.54 ± |
| | thyrocytes | 1.81 | 3.92 | 2.72 | 2.82 | 0.00 | 2.31 |
| | per 48 hours | PASS | FAIL | PASS | PASS | FAIL | PASS |
| | on day 14 | | | | | | |
| T3 levels | ng T3/10⁴ | LOT A | LOT B | LOT C | LOT A | LOT B | LOT C |
| | thyrocytes* | 16.34 ± | 13.86 ± | 45.48 ± | 17.10 ± | 8.48 ± | 34.78 ± |
| | per 48 hours | 1.90 | 4.54 | 5.34 | 1.71 | 0.66 | 3.89 |
| | on day 14 | | | | | | |

*Value only reported - no pass/fail criteria

Historical practice for preparation of this and similar media for supporting hormonogenic cells has been to prepare each element from individual stocks (separately) and combine only at the time of use. Freezing of multiple components together can be challenging; sometimes they require different diluents for solubilization, or they may precipitate out during freeze/thaw. At times, some components have varying stability based on storage temperature and relative concentrations. According to the present invention, a combination of supplements was derived that could be frozen and thawed without precipitation or loss of performance in cell-based assays, as shown above.

### Example 16: Microtissue Size and T4 Production

Microtissue size and T4 production were measured after 14 days of 3D culture in human thyrocytes that were generated and cultures across multiple methods (OG, NxtGen, p0, NG-AOPI). Regardless of the method used to generate the thyrocytes, or whether the thyrocytes were assessed at or after cryopreservation at p0 or at p1, or whether the thyrocytes were stimulated with 1.0 or 0.3 mIU/mL TSH, there was a strong correlation of microtissue size to T4 production (correlation coefficient=0.8738). Note the poor or marginal or absent T4 secretion in thyrocytes that form microtissues that are less than 30 microns in size. Correlation analysis was performed in MiniTab. The results are shown in the table below. ImageJ software was used to measure microtissue size. Images were taken using the 4× objective on a BX41 microscope (Olympus, Tokyo, Japan), and then opened in ImageJ. To ensure the scaling of the microtissues, the scale bar on the image was measured through the software. The length of the microtissues were measured in μm from ≥5 images taken on days 1, 2, 5, 7, 9, 12, and 14. For each condition, ten microtissues were measured. The average size and standard deviation were calculated using Microsoft Excel.

| | | | Avg T4 |
|---|---|---|---|
| | | Day 14 | (ng T4 |
| | TSH | Microtissue | per 10⁶ thyrocytes |
| Lot | (mIU/mL) | size (μm) | after 48 hrs) |
| 2314417 | 0.3 | 36.1 | 0 |
| 2314417 | 0.3 | 32.1 | 0 |
| 2312660 | 0.3 | 41.7 | 0 |
| 2318362 | 1 | 55.9 | 2.28 |
| 2318362 | 0.3 | 29.4 | 3.26 |
| 2318362 | 1 | 27.8 | 4.28 |
| 2318362 | 1 | 38.2 | 6.01 |
| 2318362 | 0.3 | 35.9 | 6.75 |
| 2318362 | 0.3 | 36.6 | 7.29 |
| 2318362 | 0.3 | 42.7 | 15.77 |
| 2318362 | 1 | 52.4 | 17.79 |
| 2312660 | 0.3 | 53.8 | 21.53 |
| 231873 | 0.3 | 113.0 | 38.22 |
| 231873 | 0.3 | 118.7 | 74.4 |
| 2313606 | 0.3 | 115.6 | 83.56 |
| 2314417 | 0.3 | 76.3 | 97.8 |
| 2313606 | 0.3 | 143.9 | 105.21 |

### Example 17

According to the invention, this Example shows improved results by seeding an effectively higher number of cells/higher seeding density in the initial step of culture immediately after isolation, yielding the desired culture morphology and retention of T4-competence.

Original methods for culture of freshly-isolated thyrocytes prior to cryopreservation involved enumeration of viable cells using trypan blue staining to label dead cells and enumeration using a hematocytometer or cellometer. In-process observations noted significant disparity between the live cell counts obtained using this method and the actual number of viable cells that were present in the culture within the first 24 hours of seeding. According to one aspect of the invention, it appeared that trypan blue staining significantly overestimated live cell number, leading to an effective underseeding of the culture plates. Therefore, according to this aspect, the cells did not have adequate cell-cell contact and were too sparse to thrive, leading to poor survival/expansion in culture and concomitant loss of key functions like hormone production capacity. According to the invention, by using a fluorescent live/dead cell dye (AO/PI) paired with an automated cellometer, more accurate viable cell counts were obtained that eliminated the problem of effectively underseeded cultures and supported the proper density of thyrocytes to survive in culture while retaining the correct epithelial cell morphology and downstream retention of hormone production capacity. Direct same-lot comparisons of the original method and the improved method demonstrate superior functional outcomes in this example.

The following procedures were utilized:
a. Thyroid tissue was trimmed of excess adipose and connective tissue and minced aseptically.
b. Minced thyroid tissue was digested for up to 60 minutes at 37 C in a pre-warmed enzyme cocktail (Collagenase 1A @ 1.25 mg/ml; Collagenase IV @2.5 mg/ml; 0.25% w/v Trypsin; prepared in HBSS w/Ca++/Mg++. Dissociated cells were passed through a 300 micron filter and collected. (Regular observation of digestion supernatant was critical to avoid underdigestion or overdigestion, with a target of achieving a single cell:aggregate ration of at least about 29:1. Retention of both single cells and aggregates was important to maintaining health and function of isolated cells.)
c. After collection, cell suspensions were subjected to enumeration by one of two methods:
   i. Add 20 μL AOPI to a 1.7 mL microcentrifuge tube and 20 μL 0.4% Trypan Blue to a 1.7 mL microcentrifuge tube.
   ii. Gently rock or swirl the cell-containing conical tube to resuspend the cells. Use a serological pipet to gently resuspend the cells.
   iii. Add 20 μL of the cell suspension to a single AOPI-containing microcentrifuge tube. Gently tap the tube to ensure the sample for counting is thoroughly mixed.
   iv. Add 20 μL of the cell suspension to a single Trypan Blue containing microcentrifuge tube. Gently tap the rube to ensure the sample for counting is thoroughly mixed.
   v. Repeat steps i-iv two additional times.
   vi. Turn on the Vision Cellometer.
   vii. Open the Cellometer software.
   viii. Count AOPI samples.
      1. Select the “AOPI_Stromal Cells” assay from the Assay Type dropdown list.
      2. Input sample ID and dilution factor as “2”.
      3. Load 20 μL of the cell-AOPI mixture in a cell counting chamber slide.
      4. Load the slide into the instrument.
      5. Click “Preview F1 Image” to preview the fluorescent image.
      6. If necessary, adjust focus by slowly turning the focus wheel located on the right side of the instrument.
      7. Ensure that the exposure threshold is between 95-100%.
      8. Click “Count” to begin the counting process.
      9. Review results and export data.
      10. Repeat count an additional 2 times using new samples to obtain a second and third count.
   ix. Count Trypan Blue Samples
      1. Select “Trypan Blue Viability” assay from the Assay Type dropdown list.
      2. Input sample ID and dilution factor as “2”.
      3. Load 20 μL of the cell-Trypan Blue mixture in a cell counting chamber slide.
      4. Load the slide into the instrument.
      5. Click “Preview B1” to preview the brightfield image.
      6. If necessary, adjust focus by slowly turning the focus wheel located on the right side of the instrument.
      7. Click “Count” to begin the counting process.
      8. Review results and export data.
      9. Repeat count an additional 2 times using new samples to obtain a second and third count.
      10. Determine total thyrocyte number/yield, thyrocyte viability, and total viable thyrocytes/mL for the final cell suspension.
d. Using counts obtained in “c” freshly isolated thyrocytes were seeded onto tissue culture-treated flasks at 85,000 cells/cm\hat{ }2 based on either the Trypan Blue counts or the AO/PI counts. All cultures were established and maintained with the same culture medium (h7H).
e. Cultures were observed daily until reaching about 80% confluence (NOTE: thyrocyte epithelial cells tend to develop multi-layered structures in the culture and ideal confluence will have obvious gaps on the tissue culture vessel surface where no cells are present.)
f. Cells were detached using standard detachment enzymes and cryopreserved/stored in the vapor phase of liquid nitrogen.

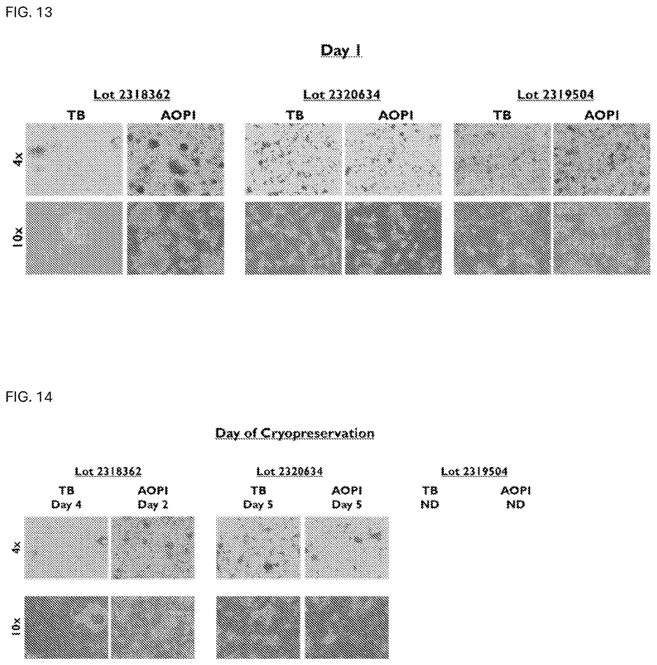

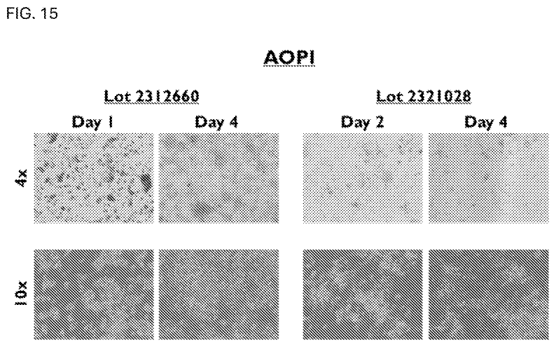

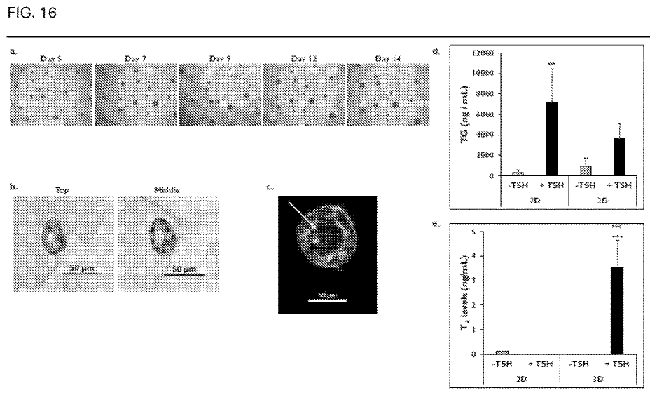

The results for this Example 17 are as follows:
a. Five thyroids were processed, and thyrocytes were isolated. One of the thyrocyte lots (lot 2319504) was contaminated. This lot could not be cryopreserved and was discarded. Lot 2318973 was accepted but was not processed due to being outside of the donor acceptance criteria (cold isochemic time CIT>24 hrs). Donor criteria is listed in the tables below.
b. Results for the total cell counts and viability post-digestion of the 5 thyroids are listed in the table below.
   i. Total cell counts and viability were consistently higher using Trypan Blue staining versus AOPI staining for all thyroids.
   ii. The cell count range using Trypan Blue staining was 17.5-133×10⁶ total cells compared to the range of 9.9-14.9×10⁶ total cells using AOPI staining.
   iii. The range of viabilities were also very different when comparing the two methods. For Trypan Blue staining, the viability range was 53-95%. AOPI staining had a lower range of between 21-77%.
c. Because of the differences in cell yield and viability between the two staining methods, cells were seeded in different size flasks (shown in the tables below). The number of flasks used also varied depending on the lot.
   iv. For lot 2318362, 2 T-182 flasks were used to seed cells after counting with Trypan Blue staining. One T-75 flask was used to seed cells after counting with AOPI staining. The seeding densities differed depending on the size of the flask used.
   v. For lots 2320634 and 2319504, flasks seeded based on Trypan Blue staining were seeded at higher cell densities compared to AOPI staining. One T-75 flask was used for each method for both lots.
d. Visual differences in cell confluency were seen in the flasks between the 2 methods. These differences on day 1 are shown in FIG. 13.
   vi. For lot 2318362, the T-75 flask using AOPI staining to seed was more confluent compared to the 2 T-182 flasks using Trypan Blue staining to seed on day 1.
   vii. The confluency was similar for both methods in lot 2320634 on day 1.
   viii. For lot 2319504, the T-75 flask using AOPI staining appeared to be more confluent compared to the T-75 flask using Trypan Blue staining on day 1.
e. Thyrocytes were cryopreserved when cells were ~80-90% confluent. The time in culture varied depending on the initial seeding density. Representative images of the cells on the day they were cryopreserved are shown in FIG. 14.
   ix. For lot 2318362, thyrocytes were cryopreserved on day 2 from the T-75 flask that was seeded using the AOPI staining method to determine seeding density. The thyrocytes seeded in the 2 T-182 flasks that used Trypan Blue to determine seeding density were cryopreserved on day 4.
   x. It should also be noted that the final cell yield from the T-75 flask (AOPI) was 27.9×10⁶ cells. The final combined cell yield from the 2 T-182 flasks was 11.6×10⁶ cells.
   xi. A similar level of confluency was seen for lot 2320634 on the day of cryopreservation (day 5) when the cells were seeded using either method to determine initial seeding density.
   xii. A similar total cell yield was measured from each flask for lot 2320634. The final cell yield from the AOPI T-75 flask was 3.35×10⁶ cells, and the yield was 2.96×10⁶ cells from the Trypan Blue flask.
   xiii. Confluency and final cell yield could not be determined from lot 2319504 due to both flasks having fungal contamination.
f. Additional thyroids were processed to isolate thyrocytes. These lots included 2312660 and 2321028.
   xiv. Post-digestion counts were performed using either Trypan Blue or AOPI staining. However, the cells were seeded at a cell density using the total cell number from the AOPI staining method only.
   xv. Representative images of cell confluency on day 1 or 2 and on the cryopreservation day are shown in FIG. 15.

Details of the thyroid donors and the thyroids are shown in the tables below.

The table below shows donor information for thyrocyte lots. All processed thyroids were within the donor specification criteria. Lot 2318973 had a CIT longer than 24 hours and was therefore not processed.

| Donor # | Age | BMI | CIT |
|---|---|---|---|
| 2318362 | 45 | 23 | 6 |
| 2320634 | 56 | 23.8 | 4.5 |
| 2319504 | 22 | 23 | 16 |
| 2312660 | 43 | | |
| 2321028 | 31 | | |

| | |
|---|---|
| 231973 | Discard: more than 24 hour CIT |

The table below shows final outcomes for processed donors. Weight of thyroid (post trimming) is included in the table. In addition, the total cell yield and viability post-digestion for each thyroid determined by either Trypan Blue (TB) staining or AOPI staining.

| | Process | Total | | | |
|---|---|---|---|---|---|
| | Tissue | Thyrocyte | % | Total | % |
| | weight | Yield × 10⁶ | Viability | Thyrocyte | Viability |
| Lot# | (g) | (TB) | (TB) | Yield × 10⁶ | (AOPI) |
| 2318362 | 11.8 | 90.2 | 90 | 9.9 | 36 |
| 2320634 | 6.0 | 22.8 | 53 | 10.1 | 29 |
| 2319504 | 3.9 | 17.5 | 56 | 9.9 | 56 |
| 2312660 | 10.6 | 54.3 | 79 | 14.9 | 21 |
| 2321028 | 7.8 | 133 | 95 | 13.0 | 77 |

The table below shows initial seeding density per flask determined by Trypan Blue staining post-digestion and final total cell yield for processed thyroids. The cells per cm2, number of flasks used, the size of the flask(s), and total number of hours in culture is also listed.

| | | Total | | | | | |
|---|---|---|---|---|---|---|---|
| | | cells | | | | Total | |
| | Counting | seeded | | | | Cell | |
| | method | per | Cells/ | Number and size | | Yield × | Hours in |
| Lot# | (seeding) | flask × 10⁶ | cm² × 10⁴ | of flask(s) | | 10⁶ | Culture |
| 2318362 | Trypan | 15.0 | 8.2 | 2 | T-182 | 11.6 | 96 |
| 2320634 | Trypan | 7.6 | 10.1 | 1 | T-75 | 2.96 | 48 |

| | | | | | | |
|---|---|---|---|---|---|---|
| 2319504 | Trypan | 7.0 | 9.3 | 1 | T-75 | Contaminated |

### 4. Discussion

a. Five thyroids were processed to isolate thyrocytes. Two cell staining methods, Trypan Blue and AOPI, were used to count cells post-digestion and then seed flasks based on the total cell count.
b. The cell counts determined by Trypan Blue staining were consistently higher and consistently had higher viabilities. Because of this, larger cell culture flasks were used to seed cells. Due to the AOPI counts and viability being consistently lower, smaller cell culture flasks were used for seeding cells. The total number of flasks used for each counting method also varied.
c. When cell confluency was examined on day 1, differences were observed. Overall, the Trypan Blue seeded flasks were visually less confluent compared to the AOPI seeded flasks even though the Trypan Blue flasks were seeded at a similar density (lot 2318362) or higher densities (lots 2320634 and 2319504).
d. There were also differences in hours in culture between the two methods. For lot 2318362, Trypan Blue flasks were seeded in larger size flasks (2 T-182 flasks) and were cultured for 96 hrs. The AOPI flask was smaller (1 T-75 flask) and cultured for 48 hours.
e. Total final cell number was measured to determine any differences between the two methods. Lot 2320634 used 1 T-75 flask after each counting method. The AOPI was not seeded as high (6.8×104 cells/cm2) compared to the Trypan Blue flask (10.1×104 cells/cm2). However, on the day of cryopreservation, the AOPI flask contained a higher number of total cells (AOPI: 3.35×106 cells vs TB: 2.96×106 cells).
f. The final thyroid that was tested was lot 2319504. Although this lot was contaminated, the images of confluency on day 1 show the AOPI flask to be more confluent even though a lower seeding density (7.9×104 cells/cm2) was used compared to the Trypan Blue flask (9.3×104 cells/cm2).
g. Two additional thyroids, lots 2312660 and 2321028, were processed to confirm the repeatability and robustness of using AOPI staining to count cells post digestion and then calculate seeding density using this number.
h. One T-175 flask was used for Lot 2312660. This lot is comparable to the 2 Trypan Blue flasks from lot 2318362. Both lots were seeded in the same size flask but an additional flask was used for lot 2318362. All the cells were in culture for 96 hours. When the cells from lot 2312660 were cryopreserved, the total cell number was 8.36×106 cells. Lot 2318362 had less total cells per flask (5.8×106 cells per flask). This suggests that the use of the AOPI staining to count cells post digestion and then seed is a better method than using Trypan Blue for counting.
i. Two T-75 flasks were seeded for lot 2321028 using AOPI staining for counting cells. Although this lot was cultured successfully, the donor's BMI (BMI=38) was noted. It should be stated that the donor criteria has been revised with the new inclusion criteria of a reduced BMI.

### 5. Conclusion

a. The use of AOPI staining to count thyrocytes post-digestion and then seed flasks according to the determined total cell number leads to a more consistent and reliable thyrocyte isolation procedure compared to when Trypan Blue is used to count cells post-digestion.

### Example 18

Modified h7H human thyrocyte culture medium (HTCM) was used for all thyrocyte culture experiments as provided above. Medium was sterile filtered through a 0.22 μm PES filter unit (ThermoFisher Scientific, Waltham, MA).

### Thyrocyte Cell Preparation

Thyrocytes from adult euthyroid donor tissues were isolated as described in Example 1.

### Thawing and Cell Culture (2D or 3D) of Cryopreserved P′1 Thyrocytes

For functionality studies, 96-well white-wall clear-bottom microplates were used (Perkin Elmer, Waltham WA). For morphological analysis, 96-well black-wall clear-bottom microplates were used (Corning, CA). All microplates were tissue culture-treated and either coated with 50 UL of Matrigel® at the concentration of 9-10 mg/mL for 3D culture or left uncoated for 2D applications. Coated microplates were incubated for 1 hour in a 37° C. CO2 to allow Matrigel® to solidify. Cryopreserved human thyrocytes were thawed in a 37° C. water bath for ≥90 seconds before being added dropwise into 14 ml of human thyrocyte plating medium (HTPM), which is HTCM without bovine TSH. The cell suspension was centrifuged for 5 minutes at 200×g in a swing bucket rotor. The pellet was resuspended in 1 mL of fresh plating medium, stained with 0.4% Trypan Blue (Gibco, Billings, MT) and counted using an automated cell counting system TC-20 (BioRad, Hercules, CA) or Vision automated cell counter (Nexcelom, Lawrence, MA). Cells were further diluted with HP™ to the appropriate final volume and 100 μL was plated per well at the final cell seeding densities specified in the results section. Microplates were then incubated in a 37° C. CO₂ incubator. On day 2, cell culture medium was replaced with HTCM containing bovine TSH (Sigma-Aldrich, St. Louis, MO) at the concentration indicated for each data set and maintained in the same media until day 14 with media exchange on day 5, 7, 9, and 12.

### Hematoxylin and Eosin Staining

For H&E staining, microtissues on day 12 were fixed with 10% formalin prior to processing and paraffin embedding. 4 μm cross sections were cut, deparaffined, and stained with Gill 2 Hematoxylin and Eosin Y (Richard Allan Scientific, San Diego, CA) for 2 and 3 minutes respectively. Slides were dehydrated and imaged using a Ziess Observer Z1 microscope (Zeiss, Dublin, CA).

### Immunofluorescent Staining

For CK7 and FSP1 staining, the following method was used. Thyrocytes were seeded at 100,000 cells/chamber on an 8-well chamber slide (ThermoFisher Scientific, Waltham, WA). After 18-24 hours, the medium was aspirated and 250 μL of Cytofix/Cytoperm solution (BD Biosciences, San Jose, CA) was added to each well followed by a 30 minute incubation at 4° C. for cell fixation. Samples were then washed 2 times with 250 μL of 1× Perm/Wash buffer (BD Biosciences). Primary antibodies were added including CK7 (Clone OV-TL, Agilent Dako, Santa Clara, CA) (1:100 dilution) and FSP1 (Abcam, Boston, MA) (1:100 dilution), and incubated overnight at 4° C. After overnight incubation, samples were washed with 1× Perm/Wash buffer followed by the addition of Goat Anti-mouse Alexa 488 IgG (H+L) (1:500) (Life Technologies, Carlsbad, CA) or Goat anti-rabbit Alexa-555 IgG (H+L) (1:500) (ThermoFisher). The chambers were then incubated at room temperature for 1 hour in the dark. Samples were washed with 1× Perm/Wash buffer and followed by a wash with 1×DPBS (ThermoFisher Scientific). Finally, two drops of Fluoromont G with DAPI (ThermoFisher, Waltham, WA) were added into each chamber prior to imaging. Images were acquired and processed using Ziess Observer Z1 fluorescent microscope with Apotome-2 and Zen Image Acquisition Software.

For Phallodin staining, the following method was used. Thyrocytes were seeded at 7,500 cells/well in 3D culture for 9 days. On day 9, microtissues were fixed with Cytofix/Cytoperm as described above, and primary antibody, Alexa Fluor 488 Phalloidin (ThermoFisher Scientific) (1:200 dilution), was added. After a 2 hour incubation period at room temperature in the dark, samples were washed and stained with 300 nM DAPI (ThermoFisher Scientific) for 1 hour in the dark at room temperature. The cells were then washed with 1×DPBS, and Fluoromont G with DAPI was added to each sample. Images were acquired and processed as described above using 100× magnification on the Ziess Observer Z1 microscope.

### Enzyme-linked Immunosorbent Assays (ELISA)

Human TG and T4 detection and quantification were carried out using the EHTG and the EIAT4C ELISA kits (ThermoFisher Scientific). Assays were performed according to the manufacturer's instructions using medium collected from cell culture at day 7 for TG and day 14 for T4 ELISA. A CLARIOStar (BMG Labtech) microplate reader was used for data collection. Medium collected for TG assay was diluted at a ratio of 1:200 for 2D cultures and 1:100 to 1:200 for 3D cultures. Medium collected for T4 assay was undiluted. MARS (version 3.31) software was used for data analysis according to the manufacturer's manual.

### Flow Cytometry

Anti-human Epithelial Cell Adhesion Molecule (EpCAM) (CD326, clone: VU-1D9)-FITC (Stem Cell Technologies, Vancouver, BC), anti-human CD90 (Thy-1, clone: 5E10)-Alexa Fluor 700 (ThermoFisher Scientific), and anti-human CD144 (clone: 55-7H1)-PerCPCy5.5 (BD Biosciences) were used for thyrocyte staining at 1:100 dilution. After viability and cell count were determined post-thaw, cells were centrifuged at 250×g for 5 minutes at room temperature, and the supernatant was discarded. Cell pellet was resuspended in 600 μL of BD Pharmingen Stain Buffer (BD Biosciences), and 100 μL was aliquoted into labelled FACS tubes for unstained, FMO, and full stain samples appropriately. UltraComp eBeads™ Plus Compensation beads (ThermoFisher Scientific) were prepared for single stain compensation controls. The bead vial was vortexed for 30 seconds and 1 drop was added into each labeled control FACS tube. Each tube was vortexed for 30 seconds to mix then incubated for 30 minutes at 4° C. in the dark. Attune Nxt Flow Cytometer (ThermoFisher Scientific) was used for data acquisition and De Novo Software—FCS Express 7 RUO Edition (7.12.0005) was used for data analysis. Between 5,000-10,000 events per single stain control were recorded. ≥50,000 events were recorded per sample. Cells of interest were identified and gated based on forward side scatter area (FSC-A) against the side scatter area (SSC-A) from which single cells were gated using FSC-A and forward side scatter height (FSC-H). Cells positive for the markers of interest were identified based on the gating of each marker against SSC-A. Background fluorescence and gating boundaries were determined using unstained and FMO control tubes.

### Measuring Microtissue Size

ImageJ was used to measure microtissue size. Images were taken using the 4× objective on a BX41 microscope (Olympus, Tokyo, Japan), and then opened in ImageJ. To ensure the scaling of the microtissues, the scale bar on the image was measured through the software. The length of the microtissues were measured in μm from ≥5 images taken on days 1, 2, 5, 7, 9, 12, and 14. For each condition, ten microtissues were measured. The average size and standard deviation were calculated using Microsoft Excel.

### Reference Chemical Evaluation

6-propyl-2-thiouracil (CASRN 51-52-5) and Methimazole (CASRN 60-56-0) were obtained from MilliporeSigma (Burlington, MA). 3D cultures with cell seeding density of 7,500 cells/well were treated with 1 mIU/mL bovine TSH on days 2, 5, 7, 9, and 12. For each studied compound, cells were dosed on days 9 and 12 with concentrations ranging from 0.0001 to 100 μM across a log-fold titration. T4 levels were measured as described. All measured data were normalized to DMSO vehicle control.

### Cell Titer Glo Assay

After collecting media for T4 measurement, backing tape was put on the back of the 3D culture 96-well plate. 100 μL of a 1:1 mixture of CellTiter-Glo® Luminescent Cell Viability Assay (Promega, Madison, WI) and HTCM was added into each well of the 3D cell culture plate. The plate was covered and shaken for 2 minutes on an orbital plate shaker at 500 rpm followed by a 10 minute incubation in the dark without shaking. Luminescence reading was recorded using a CLARIOStar (BMG Labtech) microplate reader.

### Statistical Analysis

Images are shown from representative donor lots. Values were normalized to the number of cells seeded per well. Significance was calculated in MiniTab (State College, PA) using either Student's t-test between 2 groups or One-way ANOVA with Tukey post hoc testing when comparing 3 groups or more to determine statistical significance with 95% confidence and *p<0.05 for statistical significance. GraphPad Prism v.9 was used to determine EC50 and EC90. Four-parameter nonlinear regression fit was used to determine the half-maximal inhibition concentration IC50 using GraphPad Prism v.9.

### Results

### 3D Microtissue Formation in Culture

Cryopreserved p′1 primary human thyrocytes were seeded in 3D culture using Matrigel® to determine if cells would self-aggregate to form microtissues that resemble their native follicle-like structure in vivo (FIG. 16). Cells began to aggregate, starting 24 hours after seeding, and distinct individual microtissues formed by day 5 (FIG. 16A). These microtissues had a smooth, round shape with clearly defined borders throughout the 14-day culture period. The morphology of these microtissues on day 10 showed distinct follicle-like structures with empty pockets absent of cells, which can be seen from the top and middle view (FIG. 16B). Additional staining of these microtissues on day 9 showed a similar concentration of cells on the edges with “holes” in the center lacking cells (FIG. 16C).

Once microtissues formed, their functionality was measured by determining TG and T4 secretion. 2D cultures were used as a control to demonstrate sensitivity to TSH stimulation. Thyrocytes in both 2D and 3D cultures secreted TG when stimulated with TSH (FIG. 16D). Cells in 2D culture secreted more TG compared to microtissues in 3D culture (7181±3291 vs. 3658±1437 ng/ml). Cells in 2D treated with TSH secreted significantly higher levels compared to non-treated cells (327±214 ng/ml). When T4 levels were measured, microtissues in 3D culture treated with TSH secreted significantly higher levels of T4 compared to 2D culture, which secreted little to no T4 with or without TSH stimulation (3.5±1.1 vs ND ng/ml) (FIG. 16E). Microtissues formed in 3D culture are responsive to TSH and secrete TG and T4 unlike cells in 2D culture that only synthesize TG.

### Characterization of Distinct Cell Populations

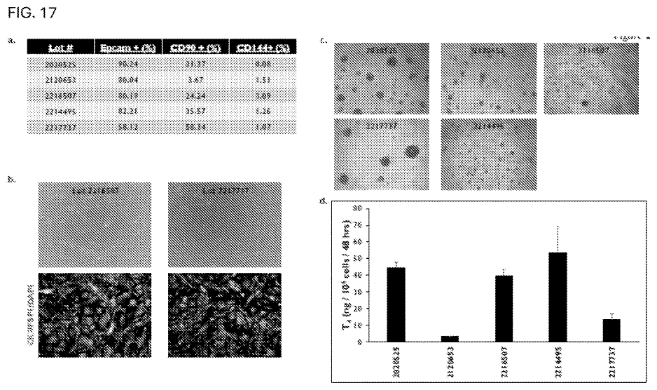

Because the formation of 3D microtissues involves the aggregation of cells, it was important to determine the types of cells forming them (FIG. 17). FLOW cytometry was performed on five donors to characterize which cell types are present when these cells are seeded in 3D culture (FIG. 17A). Three markers were used, including EpCAM, CD90, and CD144. Four of the five donors had ≥80% EpCAM positive cells. The fifth donor, 2217737, had about 58% EpCAM positive cells. This donor also had the highest number of CD90 positive cells (~58%). Donor 2120653 had the lowest number of CD90 positive cells (about 3.7%). All five donors had <5% CD144 positive cells. Protein expression of CK7 and FSP1 was performed on donors 2216507 and 2217737 (FIG. 17B). Both donors stained positive for CK7, while donor 2217737 appeared to have a distinct pattern of FSP1 compared to donor 2216507.

When these donors were seeded in 3D culture, donor 2120653 appeared to form the smallest microtissues (FIG. 17C). Each of the lots had microtissues of varying sizes and number. Donor 2217737 appeared to have the fewest number of microtissues compared to the other four donors tested. When the microtissues were stimulated with TSH, donors 2120653 and 2217737 secreted the lowest levels of T4 compared to the other donors (3.2±0.3 and 13.2±3.7 ng/10⁶ cells/48 hrs) (FIG. 17D). The T4 levels from the other donors ranged from 53.6 to 39.6 ng/10⁶ cells/48 hrs.

### Seeding Density Range for Microtissue Formation in 3D Culture

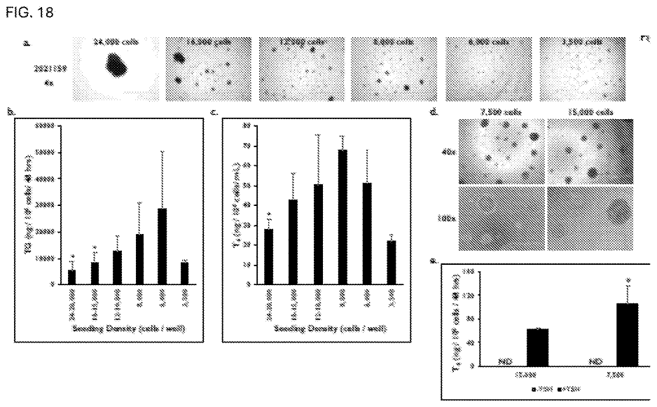

Microtissue formation in 3D culture is a necessary component for T4 secretion; therefore, the effect of the number of cells seeded per well to allow for microtissue formation was examined (FIG. 18). Cells were seeded in varying ranges from 24,000-20,000, 16,000-15,000, 12,000-10,000, 8,000, 6,000, and 3,500 cells/well (FIG. 18A). Microtissues of varying sizes and shapes formed at the different seeding densities. At the highest seeding density, 24,000-20,000 cells/well, 1 to 2 microtissues formed. These microtissues visually appeared to be larger than the other microtissues seeded at the different ranges. Microtissues appeared to decrease in size as the seeding density was lowered; however, cells were still able to self-organize into microtissues at the lowest seeding density of 3,500 cells/well in 3D culture.

TG and T₄ levels were measured from microtissues in 3D culture formed at each seeding density. As the seeding density decreased, the level of TG increased from microtissues stimulated with TSH (FIG. 18B). However, there was a drop off in TG levels (8,079±1,125 ng/10⁶ cells/48 hrs) at the lowest seeding density of 3,500 cells/well. Cells that were seeded at 6,000 cells/well formed microtissues that secreted the highest level of TG (28,399±21,870 ng/10⁶ cells/48 hrs). Significantly lower TG levels were measured from microtissues formed from cells seeded at the highest seeding densities of 24,000-20,000 (5,283±3538 ng/10⁶ cells/48 hrs) and 16-15,000 cells/well (8,151±4,224 ng/10⁶ cells/48 hrs) compared to the 6,000 cells/well seeding density.

A similar trend was seen for T₄ at the different seeding densities (FIG. 18C). As the number of cells seeded/well decreased, the level of T₄ increased. However, a drop off in secretion was seen from microtissues formed at the 6,000 and 3,500 cells/well seeding densities (51.1±16.4 ng/10⁶ cells/well and 22.2±3.0 ng/10⁶ cells/well). T₄ levels were highest from microtissues that formed when cells were seeded at 8,000 cells/well (68.0±6.8 ng/10⁶ cells/48 hrs). These levels were significantly lower from microtissues formed when cells were seeded at 24-20,000 cells/well (27.6±5.7 ng/10⁶ cells/48 hrs) compared to the 8,000 cells/well seeding density. The lowest level of T₄ measured was from microtissues seeded at 3,500 cells/well (22.2±2.9 ng/10⁶ cells/48 hrs).

As further confirmation of seeding cells at the optimized seeding density, two seeding densities were selected to test, 15,000 and 7,500 cells/well. Both seeding densities formed microtissues that visually looked similar in 3D culture (FIG. 18D). However, the microtissues formed from cells seeded at the 7,500 cells/well secreted significantly higher levels of T₄ compared to the microtissues formed from cells seeded at 15,000 cells/well when stimulated with TSH (105.2±31.28 vs. 62.6±2.8 ng/10⁶ cells/well) after normalizing to seeded cell number (FIG. 18E).

### TSH Stimulation and Differences in Microtissue Size in 3D Culture

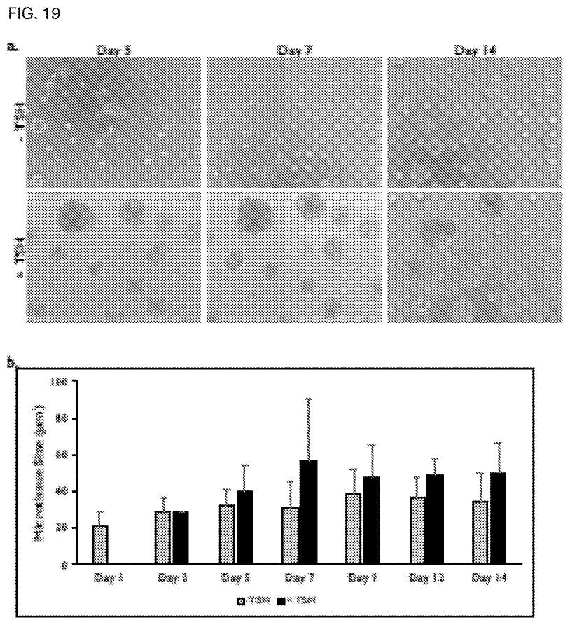

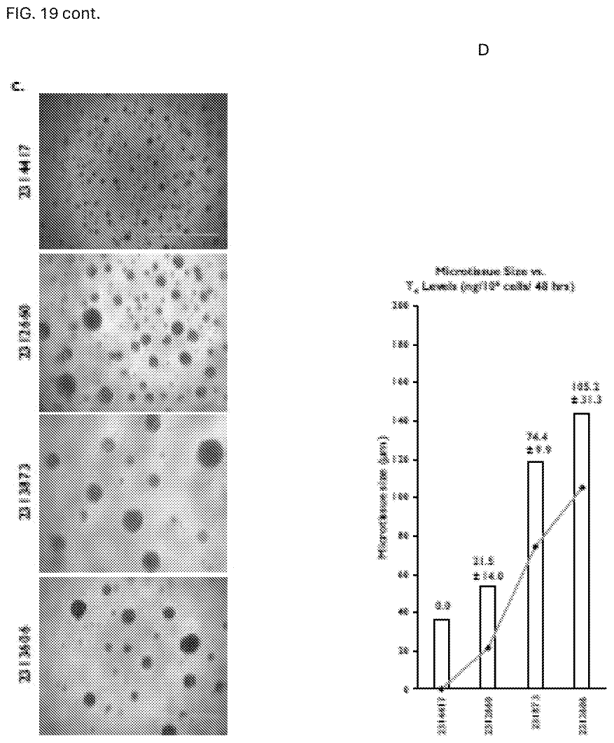

Relative differences in size were observed from non-stimulated versus TSH stimulated microtissues in 3D culture (FIG. 19). Representative images of microtissues treated without or with TSH on days 5, 7, and 14 are shown (FIG. 19A). It appears that larger microtissues are present when stimulated with TSH. To confirm this, microtissue size was determined between the 2 groups on days 5, 7, 9, 12, and 14 (FIG. 19B). Larger microtissues were measured starting on day 5 (39.8±14.8 vs. 32.4±8.8 μm), peaking by day 7 (56.0±34.5 vs. 31.7±13.4 μm), and remaining until day 14 (49.6±16.5 vs. 34.6±15.6 μm) when treated with TSH versus non-stimulated. Although there is a slight increase in size on days 1 (20.9±7.9 μm) and 2 (28.4±8.2 μm) in non-stimulated TSH microtissues, the range of size in consistently larger in the TSH stimulated microtissues.

Because microtissues increase in size due to TSH stimulation, it was tested whether larger microtissues would synthesize higher TG and T₄ levels. Microtissues in 3D culture from 4 donors were tested (FIG. 19C). There were visual differences in microtissues between each donor. Donor 2314417 appeared to form the smallest microtissues, while donor 2313606 appeared to form the largest. When microtissue size was measured, donors 2314417 and 2312660 formed microtissues averaging 36.1±3.7 and 53.8±9.7 μm (FIG. 19D). Larger microtissues were measured from donors 2313873 and 2313606 (118.7±36.7 and 143.9±42.5 μm).

T₄ was found to be correlated to microtissue size. As microtissue size increased, T₄ levels increased. No detectable quantity of T₄ was measured from Donor 2314417. The remaining donors all secreted T₄ with donor 2312660 having the lowest level (21.5±14.0 ng/10⁶ cells/48 hrs). Donors 2313873 and 2313606 secreted the highest T₄ levels (74.4±9.9 and 105.2±31.3 ng/10⁶ cells/48 hrs). These data suggest T₄ secretion may be optimal in microtissues that are at least >40 μm in size.

### TSH Concentration and T₄ Synthesis

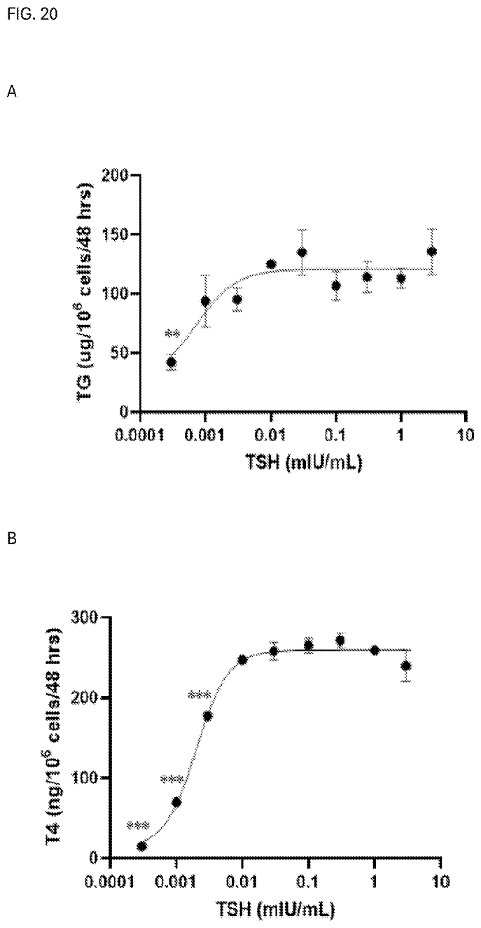

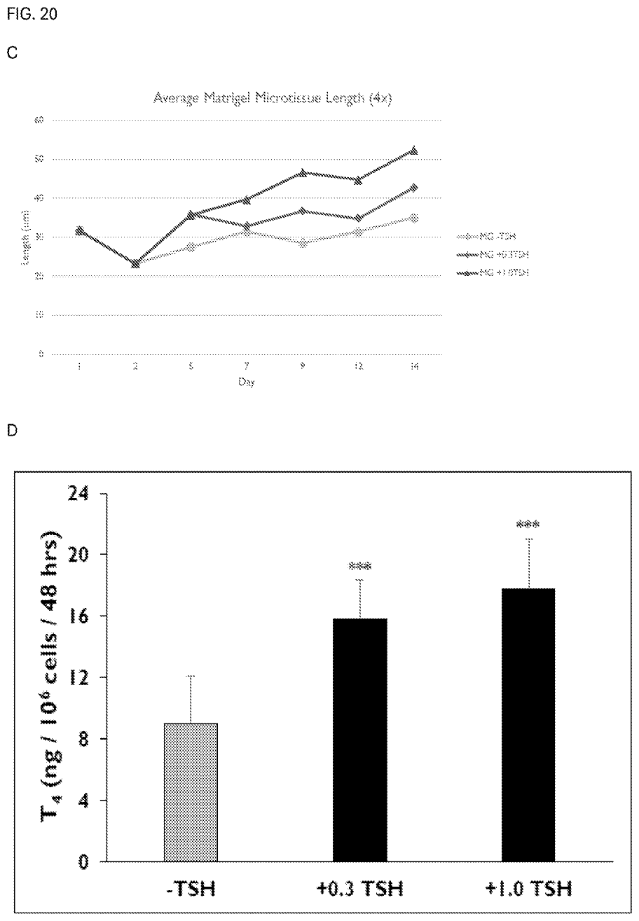

TG and T₄ synthesis from microtissues in 3D culture are dependent upon TSH stimulation. The sensitivity of the microtissues to TSH concentration was determined for each hormone (FIG. 20). Multiple TSH concentrations were tested including 0.0003, 0.001, 0.003, 0.01, 0.03, 0.1, 0.3, 1, and 3 mIU/mL. A non-stimulated control (0 mIU/mL) was also included. There were no significant differences found in TG levels when microtissues were stimulated between 0.001 mIU/ml (93.8±37.7 μg/10⁶ cells/mL) to 3 mIU/mL (135.8±33.2 μg/10⁶ cells/mL) TSH (FIG. 20A). Significantly lower TG levels were measured when microtissues were stimulated with 0.0003 mIU/mL TSH (42.2±11.7 μg/10⁶ cells/mL) compared to the 3 mIU/mL concentration. The EC₅₀ and EC₉₀ for TG secretion was 0.0008 and 0.003 mIU/mL. Non-stimulated microtissues secreted measurable levels of TG (31.7±9.5 μg/10⁶ cells/48 hrs) that were not significantly different compared the 0.0003 mIU/mL stimulated levels.

The TSH concentration range for maximal secretion of T₄ was tighter when compared to TG (FIG. 20B). No significant differences in T₄ levels were measured from microtissues stimulated with TSH 0.01 to 3 mIU/mL (247.09±2 to 239.75±39 ng/10⁶ cells/48 hrs). TSH concentration 0.3 mIU/ml (271.71±19 ng/10⁶ cells/48 hrs) stimulated the highest T₄ levels. The EC₅₀ and EC₉₀ for T₄ secretion was 0.002 and 0.007 mIU/mL. Microtissues stimulated with TSH concentrations 0.0003 (14.68±2 ng/10⁶ cells/48 hrs), 0.001 (69.69±9 ng/10⁶ cells/48 hrs), and 0.003 (177.73±11 ng/10⁶ cells/48 hrs) mIU/mL secreted significantly less T₄ compared to the higher concentration TSH (0.01, 0.03, 0.1, 0.3, 1, and 3) stimulated microtissues. Although non-stimulated microtissues produced T₄ (17.63 #1 ng/10⁶ cells/48 hrs), the level was not significantly higher compared to the those measured for the 0.0003 mIU/mL TSH stimulated microtissues.

As shown above, microtissue size increased with TSH stimulation, and T₄ levels were correlated with a minimum size of >40 μm for T₄ synthesis. In FIG. 20C, the lower line shows stimulation with MG only, no TSH. The middle line shows MG and 0.3 TSH and the upper line shows MG and 1.0TSH. After microtissues were stimulated with either 0.3 or 1 mIU/mL TSH, their size was determined on days 5, 7, 9, 12, and 14. Both TSH concentrations caused microtissues to increase in size throughout the 14-day culture period. Microtissues started at 23.3±13.5 μm on day 2 and increased in size to 32.9±18.2 and 39.7±26.4 μm on day 7 with 0.3 and 1.0 mIU/mL TSH stimulation. By day 14, TSH 0.3 and 1.0 mIU/mL stimulated microtissues increased in size to 42.7±18.4 and 52.4±41.1 μm. Non-stimulated microtissues ranged in size from 27.6±11.6 to 35.0±17.3 μm on days 5 to 14. When T₄ levels were measured, there was no significant difference in values between the concentrations used for stimulation (FIG. 20C). 0.3 and 1.0 mIU/mL TSH stimulated microtissues produced 15.8±2.6 and 17.8±3.2 ng/10⁶ cells/48 hrs, which were significantly higher compared to the levels from non-stimulated microtissues (9.0±3.1 ng/10⁶ cells/48 hrs). FIG. 20D shows microtissue size vs T₄ levels for four different donors.

### Evaluation of Thyroid-Disrupting Reference Compounds

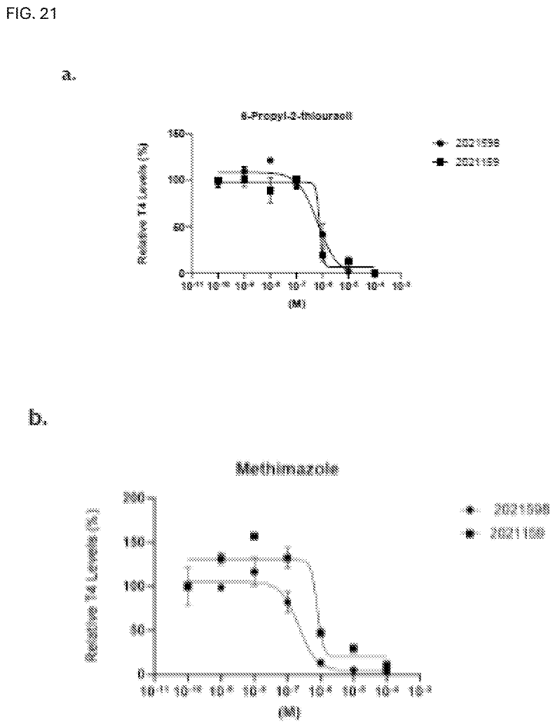

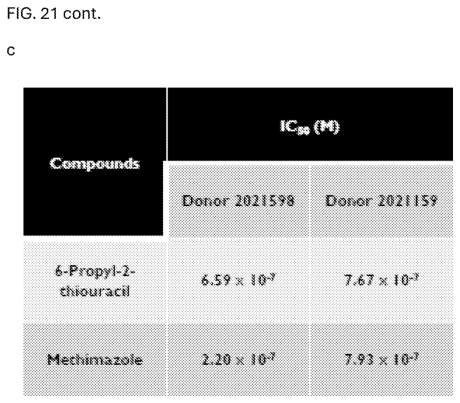

Established antagonists of thyroid hormone production were selected to determine the concentration-response profiles in 3D cultures for TDC screening purposes (FIG. 21). Inhibition of T₄ synthesis was determined after a 120-h treatment between days 9 and 14 with 6-propyl-2-thiouracil, a TPO and DIO-1 inhibitor (FIG. 21A), and methimazole, a TPO inhibitor (FIG. 21B), at log concentration intervals between 1.0×10⁻¹⁰ to 1.0×10⁻⁴ M. The concentration of inhibitors was not toxic to the cells with the exception of 6-propyl-2-thiouracil for 2021159 at the highest dose. Average half-maximal inhibitory concentrations (IC₅₀) for 6-propyl-2-thiouracil and methimazole were 6.59×10⁻⁷ M and 7.67×10⁻⁷, 2.20×10⁻⁷ and 7.93×10⁻⁷ M, respectively (FIG. 21C).

The aim of this study was to investigate the culture and treatment conditions of cryopreserved human thyroid cells at passage 1 as a potential model system for TDC testing in a 3D microtissue assay. To demonstrate that cultures of cryopreserved p′1 primary human thyrocytes can be used as a relevant model for downstream TDC screening applications, characterization of the cells and assay was performed. Determining the cell types involved in 3D microtissue formation was performed using FLOW Cytometry. Assessment of the expression of the epithelial marker EpCAM, the fibroblast marker CD90, and the endothelial marker CD144 post-thaw resulted in 4 out of the 5 lots tested having ≥80% EpCAM+ cells. The percentage of CD90+ cells in the 5 lots varied with 3 out of the 5 lots having about 30% CD90+ cells but with one lot having <5% and another lot having >50%. All 5 lots had ≤5% CD144+ cells. When these lots were stimulated with TSH, T₄ levels varied. The lot that produced the lowest level of T₄ had 80% EpCAM+ cells but <5% CD90+ cells. The second lowest T₄ producing lot had 58% EpCAM+ cells and 58% CD90+ cells. According to one aspect of the invention, a dynamic ratio of EpCAM+, CD90+, and CD144+ cells are needed to synthesize T₄. In another aspect, a relatively high number of EpCAM+ cells are needed plus a mix of CD90+ cells.

Studies were conducted to determine the relationship between cell seeding density and its impact on microtissue formation and optimal T₄ production. A similar trend of lowering cell seeding density leading to increasing TG and T₄ synthesis was observed in 3D microtissues. When normalized to total cell number/well, a peak was observed at the seeding density of 6,000 cells/well for TG production and 8,000 cells/well for T₄ production. Without wishing to be bound to any theory, this may be due to the number of microtissues that can achieve a certain critical size that are similar to that observed in vivo (approximately 50-150 microns in diameter). Confocal microscopic observations suggest that follicular-like structural features including luminal spaces surrounded by a single-layer of polarized epithelial cells of physiologically-relevant size and configuration form preferentially at specific cell densities where TG and T₄ synthesis and secretion occurs at the highest level. These studies suggest that this occurs in microtissues ≥40 μm in diameter. The higher limit of the microtissue size was not able to be determined because even the largest microtissues formed secreted quantifiable amounts of T₄. It has been found that in vivo small (range 30-100 μm) and intermediate (100-200 μm) follicles were seen to be more active than larger follicles (≥200 μm), which were thought to be hypofunctioning. Moreover, these results indicate that the preferred cell density to achieve this size range is between 6,000 and 12,000 cells per well (when using the cell counting methods described in this Example).

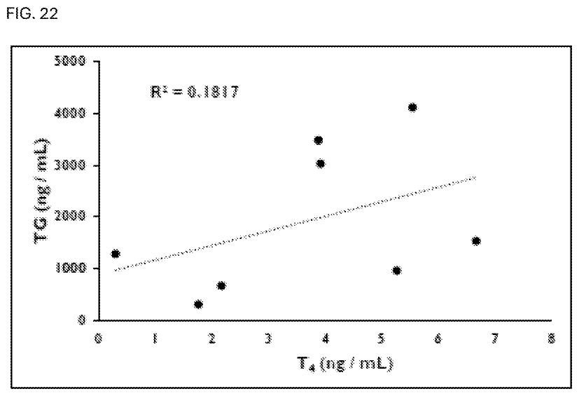

The TSH concentration-response profile for TG and T₄ production in this system were performed in 3D p′1 microtissue cultures across a wide range of TSH concentrations (0.0003 mIU-3 mIU/mL). A standard TSH concentration used to stimulate cells in vitro is 1 mIU/mL, while circulating TSH levels for healthy adults (>20 years old) are between 0.3 and 4.5 mIU/mL. Results from 3D microtissue cultures that were continuously treated with a broad range of TSH concentrations between days 2 and 14 showed TSH-induced changes in TG and T₄ production. Differences in the minimal concentration used to stimulate TG and T₄ production were seen. The lowest TSH concentration used to stimulate TG production was 0.001 mIU/mL and remained stable up to the highest concentration of 3 mIU/mL. Stable T₄ production started at 0.01 mIU/mL and remained fairly consistent up to 3 mIU/mL. Synthesis of TG may be more sensitive to TSH due to its production being directly stimulated by TSH binding to the Thyroid Stimulating Hormone Receptor (TSH-R) located on the follicle. However, T₄ production is dependent upon multiple factors, including production of T₃. Although TG levels influence synthesis of T₄ in vivo, no correlation was found between levels of TG and T₄ in these 3D microtissues (FIG. 22). Because of the more complex production pathway of T₄ and its dependence on multiple factors, higher concentrations of TSH may be required to see differences in stimulation. Overall, the results from these experiments would suggest that optimal TSH concentrations for TDC screening are between 0.01 to 3 mIU/mL.

As shown in FIG. 21, p′1 microtissue cultures were treated with known thryroid disrupting agents, methimazole (a potent TPO inhibitor) and 6-propyl-2-thiouracil (a TPO and DIO-1 inhibitor) at concentrations between 0.0001 to 100 μM. The efficacy and potency of their effects on T₄ production rates were determined. The results showed a consistent concentration-dependent decrease in T₄ levels for both inhibitors. The calculated IC₅₀ values for each compound are shown in FIG. 21. Both methimazole and 6-propyl-2-thiouracil caused complete inhibition of T₄ production at concentrations ≥10 mM. Both direct and indirect effects on downstream T₄ production and secretion rates are being measured using the 3D primay thyrocyte microtissue assay with, which is the most relevant apical outcome for thyroid disruption by chemical exposure in vivo

The results provide additional evidence for the potential contribution of this p′1 microtissue assay system for the testing of thyroid disrupting chemicals, especially for human risk assessment. Historically, there has been a lack of primary cell-based model systems for human thyroid, especially models that exhibit the key features required for a more complete approach to chemical testing, such as the physiologically relevant cellular architecture of the basic follicular substructure and the full complement of molecular and biochemical pathways pertinent to most of the key events established for the current thyroid adverse outcome pathway network. Unlike other human in vitro model systems currently available, such as microsomes and cell lines that express a limited subset of pathways, this 3D microtissue system provides substantial improvements to assess the more complex interactions that can occur simultaneously on multiple biochemical and molecular pathways (e.g. TSHR signaling, NIS transport, TPO/DIO inhibition). In addition, the system in certain embodiments establishes a new approach model and testing strategy for examining perturbations of the cellular architecture surrounding and within the colloidal space, which is the center of thyroid hormone production. Moreover, this system in certain aspects establishes a preferred method for determining the net effects of compounds on thyroid functions. For example, compound potency and efficacy data generated using simpler in vitro model systems could be put into better context relative to the contribution of each pathway to overall TH production by comparing to the corresponding data from this 3D system.

This Example provides evidence in support of the use of cryopreserved p′1 human primary thyrocytes in the human thyroid microtissue assay. Microtissue cultures of p′1 exhibit a robust increase in T₄ production that is dependent on cell seeding density, microtissue size, and TSH concentration. TSH-stimulated T₄ production in 3D microtissues is also inhibited by prototype TPO disrupting reference compounds, which is consistent with published potency and efficacy data. Finally, the results from this Example indicate the potential utility of cryopreserved p′1 primary human thyrocytes for use in the human thyroid microtissue assay and represent a model system to evaluate and prioritize potential TDCs for human health risk assessment.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A thyrocyte product comprising viable thyrocytes isolated from one or more thyroid tissues, wherein the isolated thyrocytes have been cryopreserved, the isolated thyrocytes are cultured for no more than 2 passages before being cryopreserved, the isolated thyrocytes are primary cells, and the isolated thyrocytes are positive for a biomarker selected from the group consisting of cytokeratin 7 (CK7), thyroglobulin (TG), epithelial cellular adhesion molecule (EpCAM), thyroid stimulating hormone (TSH) receptor, and thyroperoxidase (TPO) receptor.

2.-5. (canceled)

6. The thyrocyte product of claim 1, wherein at least 50% of the isolated thyrocytes exhibit an epithelial morphology when cultured on a tissue culture surface in monolayer.

7. (canceled)

8. The thyrocyte product of claim 1, wherein at least 70% of the isolated thyrocytes exhibit an epithelial morphology when cultured on a tissue culture surface are positive for EpCAM by flow cytometry or immunochemistry.

9. (canceled)

10. The thyrocyte product of claim 1 comprising 5-30% fibroblasts.

11. The thyrocyte product of claim 1, wherein 1-90% of the isolated thyrocytes are in aggregates.

12. The thyrocyte product of claim 1, wherein a ratio of single cells to aggregates is from 29:1 to 100:1.

13. The thyrocyte product of claim 1, wherein the one or more thyroid tissues are from one or more donors.

14.-15. (canceled)

16. The thyrocyte product of claim 13, wherein the human individual suffers from a thyroid disease or disorder comprising at least one of hyperthyroidism, hypothyroidism, Hashimoto's thyroiditis, postpartum thyroiditis, Graves' disease, Goiter, thyroid nodules, thyroid tumors, or thyroid cancer.

17.-18. (canceled)

19. A method for preparing a thyrocyte product, comprising:
(a) digesting one or more thyroid tissues with one or more enzymes, whereby one or more digested thyroid tissues are generated;
(b) releasing thyrocytes from the one or more digested thyroid tissues, whereby released thyrocytes are generated;
(c) seeding the released thyrocytes onto a surface in a culture vessel, whereby seeded thyrocytes are generated;
(d) growing the seeded thyrocytes in a suitable medium, whereby cultured thyrocytes are generated; and
(e) harvesting the expanded thyrocytes before the expanded thyrocytes are cultured for more than 2 passages, whereby harvested thyrocytes are generated;
(f) mixing the harvested thyrocytes with a cryopreservation medium to make a cryopreservation composition; and
(g) freezing the cryopreservation composition, whereby a thyrocyte product comprising viable thyrocytes is prepared.

20. A method for preparing a thyrocyte product, comprising:
(a) digesting one or more thyroid tissues with one or more enzymes, whereby one or more digested thyroid tissues are generated;
(b) releasing thyrocytes from the one or more digested thyroid tissues, whereby released thyrocytes are generated;
(c) mixing the released thyrocytes with a cryopreservation medium to make a cryopreservation composition; and
(d) freezing the cryopreservation composition, whereby the thyrocyte product comprising viable thyrocytes is prepared.

21. The method of claim 20, further comprising:
(e) thawing the thyrocytes in the frozen cryopreservation composition;
(f) seeding the thawed thyrocytes onto a surface in a culture vessel, whereby seeded thyrocytes are generated;
(g) growing the seeded thyrocytes in an isolation medium, whereby expanded thyrocytes are generated; and
(h) harvesting the expanded thyrocytes before the expanded thyrocytes are cultured for more than 2 passages, whereby harvested thyrocytes are generated.

22. (canceled)

23. The method of claim 19, wherein from about 4000 to about 1,000,000 live thyrocytes per/cm² are seeded as measured by counting using acridine orange (AO) and propidium iodide (PI) with an automated instrument to measure the cell viability.

24. The method of claim 19, wherein the thyrocytes are seeded in at least a 29:1 single cell:aggregate ratio onto the surface in the culture vessel.

25. The method of claim 18, wherein the culture is located within a three-dimensional space having x-, y-, and z-directions,
wherein the x- and y-directions form an x-y plane on the surface of the culture vessel and the z-direction is perpendicular to the x-y plane and the thyrocytes accumulate in the z-direction of the culture without reaching full coverage of the surface of the culture vessel in the x-y plane,
wherein the thyrocytes are grown for about 40 hours to about 6 days or until the at least 70% of the cells exhibit epithelial morphology.

26. (canceled)

27. A method for preparing a thyrocyte culture, comprising:
(a) seeding thyrocytes onto a surface in a culture vessel, wherein the thyrocytes have been isolated from one or more thyroid tissues and cryopreserved; and
(b) growing the thyrocytes in a growth medium, whereby a thyrocyte culture comprising cultured thyrocytes is prepared.

28.-30. (canceled)

31. The method of claim 27, wherein the cultured thyrocytes comprise a protein selected from the group consisting of cytokeratin 7 (CK7), thyroglobulin (TG), thyroid transcription factor 1 (NKX2-1), epithelial cellular adhesion molecule (EpCAM), thyroid stimulating hormone (TSH) receptor, and thyroperoxidase (TPO) receptor.

32. The method of claim 27, wherein the cultured thyrocytes express a gene selected from the group consisting of thyroid stimulating hormone receptor (TSHR), thyroglobulin (TG), thyroperoxidase (TPO), Sodium Iodide Symporter (SLC5A5), Pendrin (SLC26A4), DIO1 gene, DIO2 gene, Dual Oxidase 1 (DUOX1), Dual Oxidase 2 (DUOX2), Dual Oxidase Maturation Factor 1 (DUOXA1), and Dual Oxidase Maturation Factor 2 (DUOXA2) genes.

33.-37. (canceled)

38. The method of claim 27, wherein the thyrocytes form microtissues attached to the surface.

39. The method of claim 38, wherein each of the microtissues has a hollow space.

40. The method of claim 38, wherein the microtissues have a diameter of 30-200 μm.

41.-42. (canceled)

43. The method of claim 38, further comprising producing a hormone selected from the group consisting of thyroxine (T4), 3,5,30-triiodothyronine (T3), monoiodothyronine (MIT), and diiodothyronine (DIT).

44.-58. (canceled)
